(19) United States
(12) Patent Application Publication
(10) Pub. No.: US 2011/0196208 A1
(43) Pub. Date: Aug. 11, 2011

(54) ASYMETRICAL SURGICAL RETRACTOR

(75) Inventors: W. Lee Warren, Auburn, AL (US); Greg Causey, Erie, CO (US)

(73) Assignee: Lanx, Inc., Broomfield, CO (US)

(21) Appl. No.: 12/890,117

(22) Filed: Sep. 24, 2010

Related U.S. Application Data
(63) Continuation-in-part of application No. 12/399,479, filed on Mar. 6, 2009.

(51) Int. Cl. A61B 1/32

(52) U.S. Cl. 600/210

(57) ABSTRACT

Surgical retractors, retractor insertion tools, and methods of use are disclosed. The surgical retractor includes a first protrusion end, a second insertion end opposite the first protrusion end, and an opening extending through the retractor body from a first end opening located at the first protrusion end of the retractor body to a second end opening located at the second insertion end of the retractor body. The first and second end openings may have different sizes and shapes. The first and second end openings may be arranged out of alignment with each other.

### CROSS-REFERENCE TO RELATED APPLICATIONS

This application is a continuation-in-part of U.S. application Ser. No. 12/399,479, entitled SURGICAL RETRACTOR, and filed on 6 Mar. 2009, which is incorporated herein in its entirety by this reference.

### BACKGROUND

Surgical retractors have long been used by surgeons as a tool for actively separating the edges of a surgical incision or wound. In so doing, surgical retractors provide improved access to an area within the body where a surgeon is operating.

One type of known surgical retractor may generally comprise a cylindrical body having an opening passing through the middle of the cylindrical body. The opposing ends of the opening through the cylindrical body are the same size and shape, making the walls of the cylindrical body parallel to one another. When one end of the cylindrical body is inserted into a wound or incision, the wound or incision area is enlarged. This provides access for surgical tools to be inserted into the wound or incision through the opening. However, the range of mobility inside the wound or incision is limited due to the parallel walls of the cylindrical body. Furthermore, sight lines are impaired once tools are extended down into the cylindrically shaped retractor. Finally, due to the curved walls of the retractor, it is difficult keep a tool stationary when it is rested against the side of the rounded retractor walls

Another type of known surgical retractor may generally comprise a conical body having an opening passing through the middle of the conical body. The opposing ends of the opening are each circular, but one end of the opening is larger than the opposite end of the opening. In application, the larger end is inserted into the wound site or incision to thereby provide a larger working area inside the wound site or incision. However, the conical body suffers from the same inability to securely rest a tool against a wall of the retractor as the conical configuration described above. Tools inserted into the wound or incision via the conical retractor also impair visibility as described above with respect to the conical retractor. Additionally, due to the concept of a lever arm, small movements of the end of the tool protruding out of the conical retractor will lead to large movements of the end of the tool located within the wound site or incision. Precise tool movements within the wound site or the incision are, therefore, hard to accomplish using a conical retractor as described above.

### SUMMARY

The present disclosure relates generally to surgical retractors and tools for aiding in the placement of surgical retractors in incisions or wound sites. The present disclosure also relates generally to methods of placing surgical retractors in incisions or wound sites.

In one embodiment disclosed herein, a surgical retractor includes a retractor body having a first end with a first end opening, a second end opposite the first end and having a second end opening, and a pass through aperture extending through the retractor body from the first end opening to the second end opening. The first end opening may have an elongate polygonal shape. The second end opening may have an elongate curved shape. A central axis of the first end opening and a central axis of the second end opening may be radially offset from each other.

In another embodiment, a surgical retractor includes a hollow retractor body having a first end opening defined at a first protrusion end, and a second end opening defined at a second insertion end opposite the first protrusion end. The first end opening may have a polygonal shape and the second end opening may have a curved shape. The second end opening may be offset laterally and longitudinally from the first end opening.

In a further embodiment, a method of treating a patient includes making an incision in a patient, and inserting a surgical retractor into the incision. The surgical retractor includes a hollow retractor body having a first end opening defined at a protrusion end, and a second end opening defined at an insertion end opposite the protrusion end. The first end opening may have a rectangular shape and the second end opening may have an elliptical shape. The second end opening may be offset laterally from the first end opening. The method may further include inserting an instrument through the surgical retractor and into the patient, and retaining the instrument in contact with a corner of the first end opening.

Features from any of the above mentioned embodiments may be used in combination with one another, without limitation. In addition, other features and advantages of the instant disclosure will become apparent to those of ordinary skill in the art through consideration of the ensuing description, the accompanying drawings, and the appended claims.

### BRIEF DESCRIPTION OF THE DRAWINGS

FIG. 1 shows a perspective view of a surgical retractor according to an embodiment disclosed herein.

FIG. 2 shows a side view of the surgical retractor of FIG. 1.

FIG. 3 shows a top view of the surgical retractor of FIG. 1.

FIG. 4 shows a cross-sectional view of the surgical retractor illustrated in FIG. 1 taken along line 4-4 shown in FIG. 3.

FIG. 5 shows a perspective view of a surgical retractor according to an embodiment disclosed herein.

FIG. 6 shows a top view of a surgical retractor according to an embodiment disclosed herein

FIG. 7 shows a top view of the surgical retractor of FIG. 1 corresponding to FIG. 3 and showing tools deposited in the surgical retractor.

FIG. 8 shows a cut-away side view of the surgical retractor of FIG. 1 corresponding to FIG. 4 and showing a tool deposited in the surgical retractor.

FIG. 9 shows a side view of a surgical retractor insertion tool according to an embodiment disclosed herein.

FIG. 10 shows a front view of the surgical retractor insertion tool of FIG. 9

FIG. 11 shows a step of a method for inserting a surgical retractor into an incision made in a patient disclosed herein.

FIG. 12 shows a step of a method for inserting a surgical retractor into an incision made in a patient disclosed herein.

FIG. 13 shows a step of a method for inserting a surgical retractor into an incision made in a patient disclosed herein.

FIG. 14 shows a step of a method for inserting a surgical retractor into an incision made in a patient disclosed herein.

FIG. 15 shows a perspective view of a surgical retractor according to another embodiment disclosed herein.

FIG. 16 shows another perspective view of the surgical retractor of FIG. 15.

FIG. 17 shows a right side view of the surgical retractor of FIG. 15.

FIG. 18 shows a left side view of the surgical retractor of FIG. 15.

FIG. 19 shows a front view of the surgical retractor of FIG. 15.

FIG. 20 shows a rear view of the surgical retractor of FIG. 15.

FIG. 21 shows a top view of the surgical retractor of FIG. 15.

FIG. 22 shows a bottom view of the surgical retractor of FIG. 15.

FIG. 23 shows a top view of the surgical retractor of FIG. 15 with a plurality of tools positioned therein.

FIG. 24 shows a cross-sectional view of the surgical retractor of FIG. 15 taken along line 24-24 in FIG. 23.

FIG. 25 shows a cross-sectional view of the surgical retractor of FIG. 15 taken along line 25-25 in FIG. 23.

FIG. 26 shows a perspective view of a surgical retractor insertion tool assembly according to an embodiment disclosed herein.

FIG. 27 shows a step of a method for inserting the surgical retractor into an incision made in a patient disclosed herein.

FIG. 28 shows a step of a method for inserting the surgical retractor into an incision made in a patient disclosed herein.

FIG. 29 shows another step of a method for inserting the surgical retractor into an incision made in a patient disclosed herein.

FIG. 30 shows another step of a method for inserting the surgical retractor into an incision made in a patient disclosed herein.

FIG. 31 shows another step of a method for inserting the surgical retractor into an incision made in a patient disclosed herein.

FIG. 32 shows a front perspective view of a surgical retractor placement tool according to an embodiment disclosed herein.

FIG. 33 shows a rear perspective view of the surgical retractor placement tool of FIG. 32.

FIG. 34 shows a front view of the surgical retractor placement tool of FIG. 32.

FIG. 35 shows a front view of an alternative surgical retractor placement tool.

FIG. 36 depicts a side view of the surgical retractor placement tool of FIG. 32.

FIG. 37 depicts a front perspective view of a surgical retractor placement tool according to an embodiment disclosed herein.

FIG. 38 depicts a front view of the surgical retractor placement tool of FIG. 37.

FIG. 39 depicts a retractor engaging a placement tool according to an embodiment disclosed herein.

Throughout the drawings, identical reference characters and descriptions indicate similar, but not necessarily identical, elements. While the exemplary embodiments described herein are susceptible to various modifications and alternative forms, specific embodiments have been shown by way of example in the drawings and will be described in detail herein. However, the exemplary embodiments described herein are not intended to be limited to the particular forms disclosed. Rather, the instant disclosure covers all modifications, equivalents, and alternatives falling within the scope of the appended claims.

### DETAILED DESCRIPTION

The instant disclosure relates generally to surgical retractors, surgical retractor insertion tools, and methods for inserting surgical retractors into an incision made in a patient. At least some of the surgical retractors disclosed herein generally employ a configuration wherein the opening of the retractor inserted into the patient is smaller than the opening protruding out of the patient. In one embodiment of the surgical retractor disclosed herein, the configuration is further defined as having a curved opening inserted into the patient and a polygon-shaped opening protruding out of the patient. A curved opening is defined as one having at least one radius and at least one center point about which the curve is defined. Exemplary curves include, but are not limited to, circles, ellipses, parabolas, hyperbolic curves, pear-shaped curves, egg-shaped curves, multi-lobed curves, or any other curve. A polygon-shaped opening may have any number of sides. For example, a polygon-shaped opening may have from three to eight sides. They may be convex or non-convex. For example, a polygon is convex if any line drawn through the polygon (and not tangent to an edge or corner) meets its boundary exactly twice, and non-convex if a line may be found which meets the boundary of the polygon more than twice. Exemplary polygon shapes include, but are not limited to, triangles, squares, rectangles, pentagons, hexagons, heptagons, octagons, and stars having any number of points. Curved and polygon-shaped openings may be open or closed, regular or irregular, symmetrical or asymmetrical. In some embodiments, one or both openings include portions which are curved and other portions which are polygon-shaped.

This configuration provides numerous benefits over prior art retractors, including the ability to securely position a tool using a corner of the polygon-shaped opening, improved sight lines when tools are inserted into the retractor, and advantageously utilizing the concept of a lever arm, wherein larger movements of the protruding end of a tool will result in smaller movements of the end of the tool inside the incision or wound. Such configurations also may permit increased angulation of tools within the patient, so a desired angle of approach to the surgical site may be used.

The retractors, surgical retractor insertion tools, and methods of this disclosure may be used for surgery in any portion of a patient's body including, but not limited to, the head, neck, chest, abdomen, joints, and other portions of the body. For example, they may be used in surgery on the human spine. Any tools may be inserted through the retractor including, but not limited to, osteotomes, burrs, reamers, forceps, scalpels, chisels, lights, suction devices, irrigation devices, implants, drivers, and other tools.

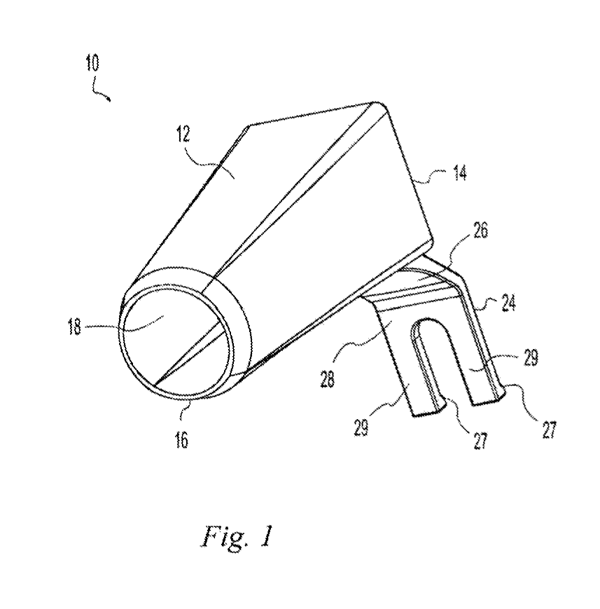

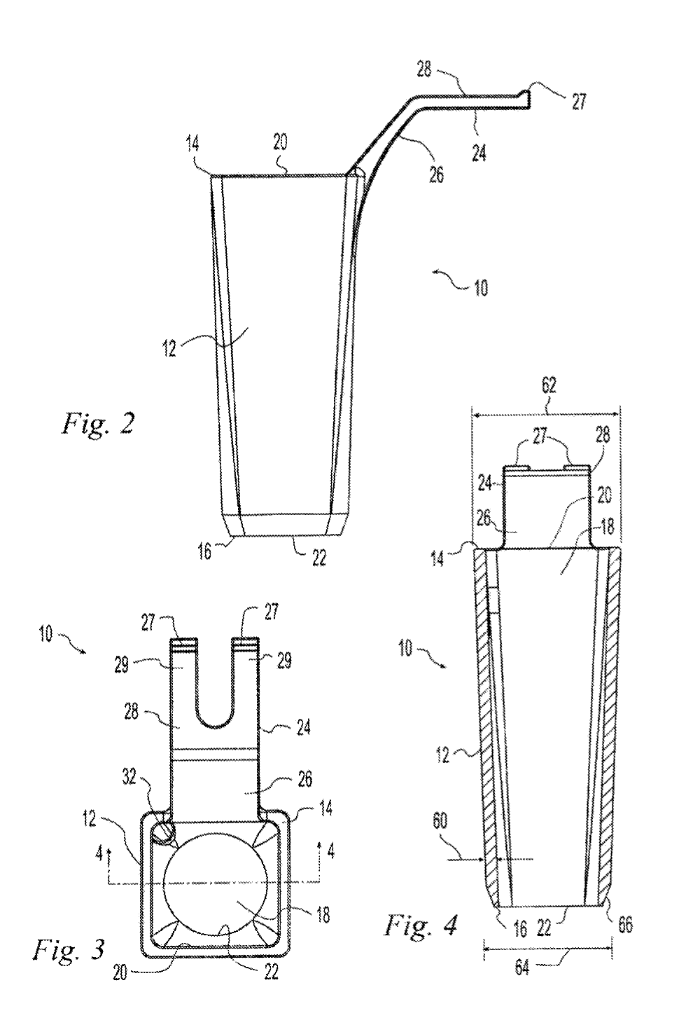

As shown in FIGS. 1-4, the surgical retractor 10 may generally comprise a retractor body 12 having a first protrusion end 14 and a second insertion end 16 opposite first protrusion end 14. Surgical retractor 10 may also comprise an opening 18 that extends through the entire length of retractor body 12 (i.e., from first protrusion end 14 to second insertion end 16). Because opening 18 extends through the entire length of the retractor body 12, opening 18 may comprise a first end opening 20 located at first protrusion end 14 of retractor body 12 and a second end opening 22 located at second insertion end 16 of retractor body 12. First end opening 20 may be larger than second end opening 22 such that opening 18 is defined by a generally conical shape that tapers from first end opening 20 to second end opening 22. In this embodiment the retractor body has a generally constant and relatively thin wall thickness 60 such that the retractor body 12 mimics the shape of the opening 18 and is a generally conical shape that tapers from a first width 62 to a second width 64. In other embodiments, the opening 18 may define one shape while the retractor body 12 defines another, different shape. The retractor body includes a chamfer 66 to ease insertion of the retractor through an incision.

In application, surgical retractor 10 as shown in FIGS. 1-4 may be inserted into a wound or incision made in a patient to open up the wound or incision area and provide improved access to and visibility of the wound or incision area. Surgical retractor 10 may be designed such that second insertion end 16 serves as the insertion end and first protrusion end 14 serves as the portion of the surgical retractor that protrudes out of the patient. Retractor body 12 keeps the edges of the wound or incision spread apart to allow for access into the wound or incision area. That is to say, upon insertion, the edges of the wound or incision rest against the exterior of retractor body 12 and are retained apart by the retractor body 12. Once in place, opening 18, including first end opening 20 and second end opening 22, provides the pathway for viewing inside the wound or incision as well as for inserting surgical tools into the wound or incision. Such surgical tools may generally be introduced into the wound or incision by passing the tools through first end opening 20 and down opening 18 to second end opening 22.

As best seen in FIGS. 1 and 3, first end opening 20 and second end opening 22 may have specific shapes designed to improve the usefulness of surgical retractor 10. Specifically, first end opening 20 may have a polygon shape and second end opening 22 may have a curved shape. While FIGS. 1 and 3 illustrate a square-shaped first end opening 20 and a circular shaped second end opening 22, any type of polygon or curve may be used.

While polygon shapes are described above for first end opening 20, the shape of first end opening 20 is not limited. First end opening 20 may have a circular shape or a shape utilizing both straight segments and curved segments. Likewise, while curved shapes are described for second end opening 22, the shape of second end opening is not limited. Second end opening 22 may have a polygon shape or a shape utilizing both straight segments and curved segments.

When first end opening 20 comprises a polygon shape, surgical retractor 10 may include corners in the proximity of first protrusion end 14. For example, as shown in FIG. 3, the square shape of first end opening 20 provides four corners at first protrusion end 14 of surgical retractor 10. Where second end opening 22 has a circular shape, these corners will gradually transition to the circular shape near second insertion end 16 (as shown in e.g., FIG. 3), but corners will remain at first protrusion end 14 of surgical retractor 10.

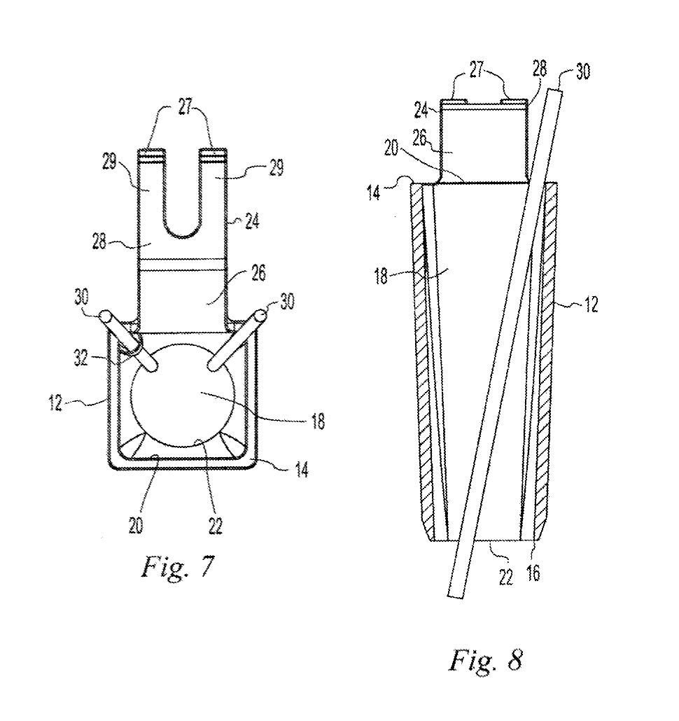

As shown in FIGS. 7 and 8, such corners may be used for resting surgical tools 30 not in use or which need to be held in a certain position while other tools are being used (such as, e.g., a light). The tools will tend to settle or be constrained in the corners such that the corners will hold tools 30 in place, unlike rounded edges which would allow tools 30 to move freely along the curved sides. To further maintain tools 30 in place, the corners may include a tool retention feature such as tool retention loop 32 near first protrusion end 14 of surgical retractor 10. Tool retention loops 32 may generally comprise loops that extend across a corner and allow tools 30 to be inserted through the loop. The size of tool retention loops 32, including how much room is provided within the loop, is not limited. Tool retention loops 32 may be large enough to accept a variety of tools having different sizes. In one aspect, tool retention loops 32 may be adjustable. For example, tool retention loops 32 may be tightened to better secure a smaller tool, or may be enlarged to accommodate a larger tool. Any number of tool retention loops 32 may be provided, including a single tool retention loop 32 or a tool retention loop 32 for every corner of the polygon-shaped first end opening 20. The tool retention loop 32 may open radially in the form of a clip that allows the tool to engage the tool retention loop 32 radially in, for example, a snap-fitting relationship.

Surgical retractor 10 may further comprise a mounting bracket 24. Mounting bracket 24 may be used to secure surgical retractor 10 to a frame or to other equipment used when surgical retractor 10 is in place in a wound or incision. Mounting bracket 24 may generally be coupled to retractor body 12 at any location along retractor body 12. In one aspect, mounting bracket 24 is coupled to retractor body 12 at a location proximate first protrusion end 14 of retractor body 12. Mounting bracket 24 may generally protrude away from retractor body 12 and also may protrude away from second insertion end 16 of retractor body 12. Mounting bracket 24 may be coupled to retractor body 12 using any suitable mechanism, such as glue, welding, bolts, or screws. In one aspect, mounting bracket 24 may be integrally formed with retractor body 12 such that surgical retractor 10 is one unitary piece.

When first end opening 20 of surgical retractor 10 has a polygon shape, mounting bracket 24 may be coupled to surgical retractor 10 at a side of retractor body 12 formed by the polygon shape of first end opening 20 as shown in FIG. 3, for example. Alternatively, mounting bracket 24 may extend over a corner of the polygon shape of first end opening 20 as shown in FIG. 6, for example. FIGS. 1-4, 7 and 8 illustrate the configuration where mounting bracket 24 may be coupled to retractor body 12 at a side of retractor body 12 as formed by the polygon shape of first end opening 20. The polygon shape of first end opening 20 as generally shown in the FIGS. is a square. Mounting bracket 24 may be coupled to any one of the four sides of the square shape or any one of the corners. Where the polygon shape of first end opening 20 has between three and eight sides, mounting bracket 24 may be coupled to retractor body 12 on any of the three to eight sides or the associated corners between the sides. FIG. 6 illustrates the configuration where mounting bracket 24 may be coupled to retractor body 12 by extending over a corner of the polygon shape of first end opening 20.

The shape of mounting bracket 24 may be any suitable shape for allowing mounting bracket 24 to be secured to a frame or other equipment used when surgical retractor 10 is in place. As shown in FIGS. 1-8 of this disclosure, mounting bracket 24 may generally comprise an angled section 26 coupled to retractor body 12. Angled section 26 may generally protrude away from both second insertion end 16 and retractor body 12 of surgical retractor 10. As shown in FIGS. 1-8, angled section 26 may generally be at a 45 degree angle to an axis running through opening 18 of surgical retractor 10, although other angles may also be used. Mounting bracket 24 may further comprise a transverse section 28 that is coupled directly to angled section 26. Transverse section 28 may generally be transverse to the axis running through opening 18 of surgical retractor 10. As also shown in FIGS. 1, 3, 6 and 7, transverse section 28 may comprise two arms 29 with a slot located therebetween. The slot may serve as the area where surgical retractor 10 may be secured to a frame or other equipment used in conjunction with surgical retractor 10. One or both arms 29 may also include a rib or boss 27 at the end of arms 29.

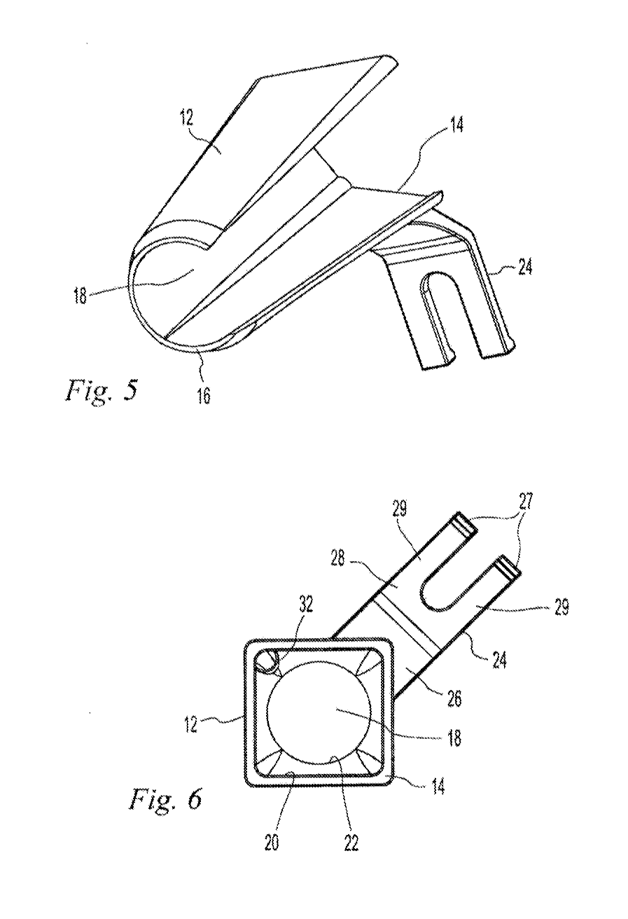

Referring now to FIG. 5, retractor body 12 may extend less than 360 degrees around opening 18. In other words, retractor body 12 may include a gap that extends from first protrusion end 14 to second insertion end 16 such that the retractor body 12 is not a closed shape. Such a configuration may further improve visibility inside the incision or the wound when surgical retractor 10 is inserted therein.

The dimensions of surgical retractor 10 are not limited and may be adjusted depending on the size of the wound or incision with which surgical retractor 10 is being used. As noted above, one limitation on dimensions may be that first end opening 20 is larger than second end opening 22. A first end opening 20 that is larger than a second end opening 22 may generally mean that retractor body 12 tapers inwardly between first end opening 20 and second end opening 22. The material of surgical retractor 10 is also not limited. In one aspect, surgical retractor 10 may comprise biocompatible material. Different parts of surgical retractor 10 may be made of the same or different materials. For example, mounting bracket 24 may be the same or a different material than the material of retractor body 12.

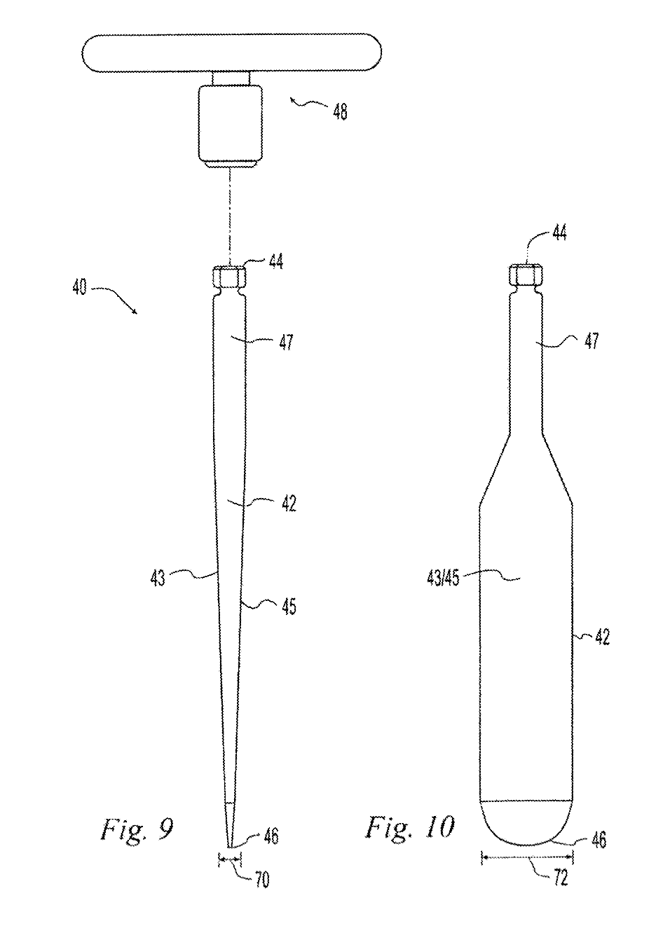

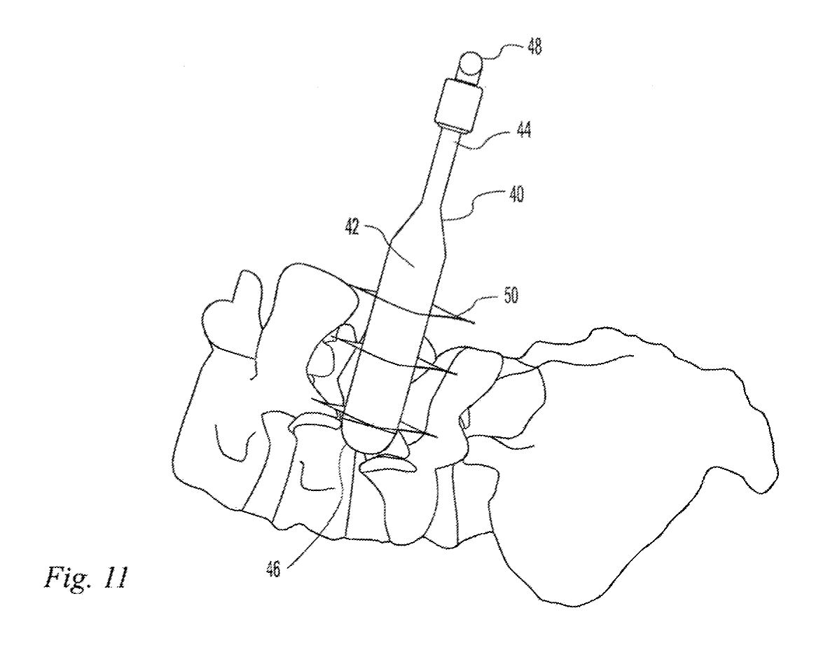

In another embodiment, a surgical retractor insertion tool that may be used to place a surgical retractor in a wound or incision is disclosed. FIGS. 9 and 10 illustrate a surgical retractor insertion tool 40. Surgical retractor insertion tool 40 may generally comprise a paddle-shaped spreader 42. Paddle-shaped spreader 42 may generally comprise a first end 44 and a second end 46 opposite first end. At first end 44 of paddle-shaped spreader 42, surgical retractor insertion tool 40 may comprise a handle 48. Handle 48 may be removably coupled to first end 44 of paddle-shaped spreader 42.

In application, surgical retractor insertion tool 40 may be inserted into a wound or incision such that surgical retractor insertion tool 40 is generally parallel with the wound or incision. Ideally, the surgical retractor insertion tool 40 is inserted such that paddle-shaped spreader 42 fits between muscle fibers (not specifically shown). Once inserted, surgical retractor insertion tool 40 may be rotated about a center axis of surgical retractor insertion tool 40. Rotation of surgical retractor insertion tool 40 may be accomplished using handle 48. Once surgical retractor insertion tool 40 is rotated to a position no longer parallel with the wound or incision, the width of paddle-shaped spreader 42 may spread apart the edges of the wound or incision, thereby making it easier to insert a surgical retractor.

The paddle shape of paddle-shaped spreader 42 may generally comprise a first broad face 43 opposite a second broad face 45, wherein the thickness 70 between first broad face 43 and second broad face 45 is small relative to the width 72 of first broad face 43 and second broad face 45. In this manner, paddle-shaped spreader 42 may resemble a paddle or oar of a boat. As shown in FIGS. 9 and 10, first broad face 43 and second broad face 45 of paddle-shaped spreader 42 may extend from second end 46 to a point short of first end 44. At this point, paddle-shaped spreader 42 may transition to a neck portion 47 that extends to first end 44. The width at neck portion 47 of paddle-shaped spreader 42 may be closer to or equal to the thickness 70 between first broad face 43 and second broad face 45. In fact, neck portion 47 of paddle-shaped spreader 42 may be cylindrical. Alternatively, first broad face 43 and second broad face 45 may extend all the way from second end 46 to first end 44 of paddle-shaped spreader 42.

The shape of first broad face 43 and second broad face 45 is not limited. As shown in FIG. 10, first broad face 43 and second broad face 45 may generally have a triangular shape at an end closest to first end 44 where paddle-shaped spreader 42 transitions from neck portion 47 to first broad face 43 and second broad face 45. First broad face 43 and second broad face 45 may then transition into a rectangular shape, wherein the edges of first broad face 43 and second broad face 45 are aligned in parallel, followed by a round-shaped edge at second end 46 of paddle-shaped spreader 42. Many other shapes and combination of shapes may also be used. For example, the entirety of first broad face 43 and second broad face 45 may have a rectangular shape, an oval shape, a diamond shape, a triangular shape, or an inverted triangular shape. First broad face 43 and second broad face 45 may also comprise any combination of the above shapes.

The thickness between first broad face 43 and second broad face 45 of paddle-shaped spreader 42 need not remain constant between first end 44 and second end 46. As shown in FIG. 9, the thickness 70 between first and second broad faces may either be maintained constant or decrease between first end 44 and second end 46. For example, the thickness 70 may remain constant near first end 44 and then transition to a constant rate of decreasing thickness as second end 46 is approached. In one aspect, the thickness 70 may decrease as second end 46 is approached such that first broad face 43 and second broad face 45 meet at second end 46 to thereby form a wedge-shaped second end 46. Such a configuration aids in inserting second end 46 into a wound or incision when using surgical retractor insertion tool 40.

Handle 48 may be removably coupled to first end 44 of paddle-shaped spreader 42. Handle 48 may be removable in order to facilitate insertion of a surgical retractor into the wound or incision after surgical retractor insertion tool 40 has been inserted and rotated to spread apart the edges of the wound or incision. Any mechanism for removably coupling handle 48 to first end 44 of paddle-shaped spreader 42 may be used. Examples include, but are not limited to, an interference fit between handle 48 and first end 44, having handle 48 screw on to first end 44, or using latches between first end 44 and handle 48 such as a ball and groove coupling. The shape of handle 48 is also not limited. As shown in FIG. 9, handle 48 may have a generally T-shape, but other shapes may be used. Handle 48, when coupled to paddle-shaped spreader 42, may be oriented in any manner with respect to paddle-shaped-spreader 42. For example, handle 48 may be perpendicular to or parallel with first broad face 43 and second broad face 45 of paddle-shaped spreader 42.

In another embodiment, a method for inserting a surgical retractor into an incision made in a patient is disclosed. The surgical retractor 10 may simply be inserted directly into an incision by pressing second insertion end 16 into the incision. The second insertion end 16 may include a chamfer 66 to aid the insertion.

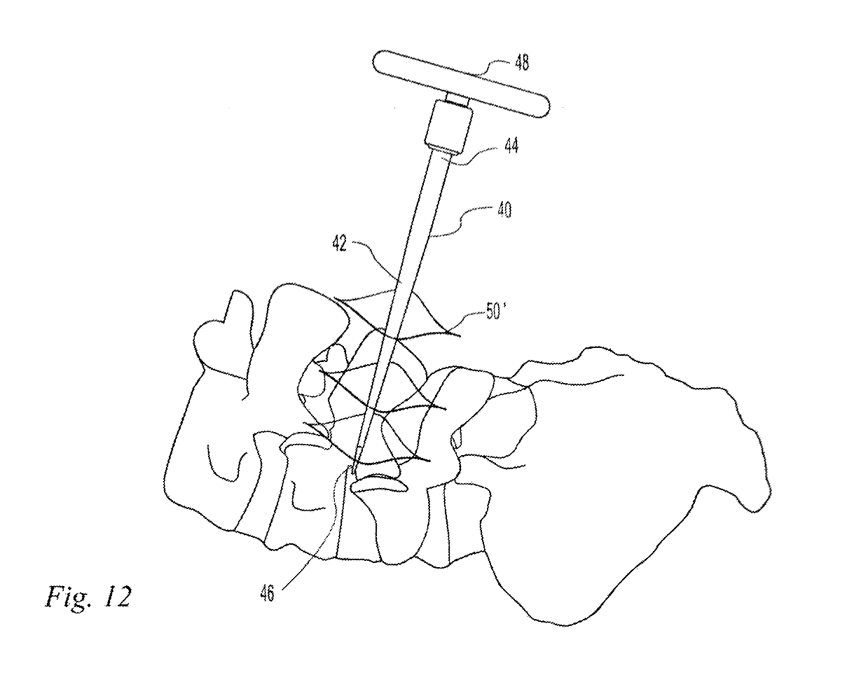

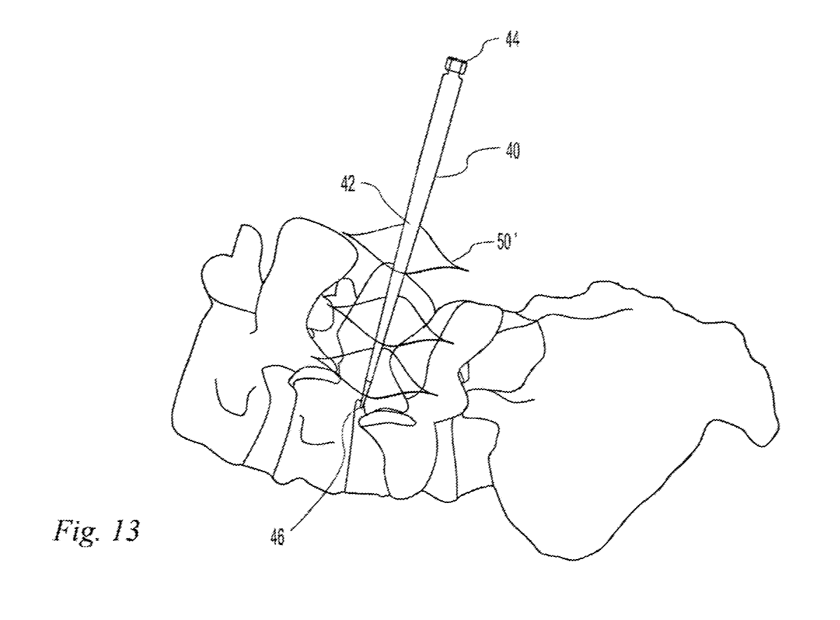

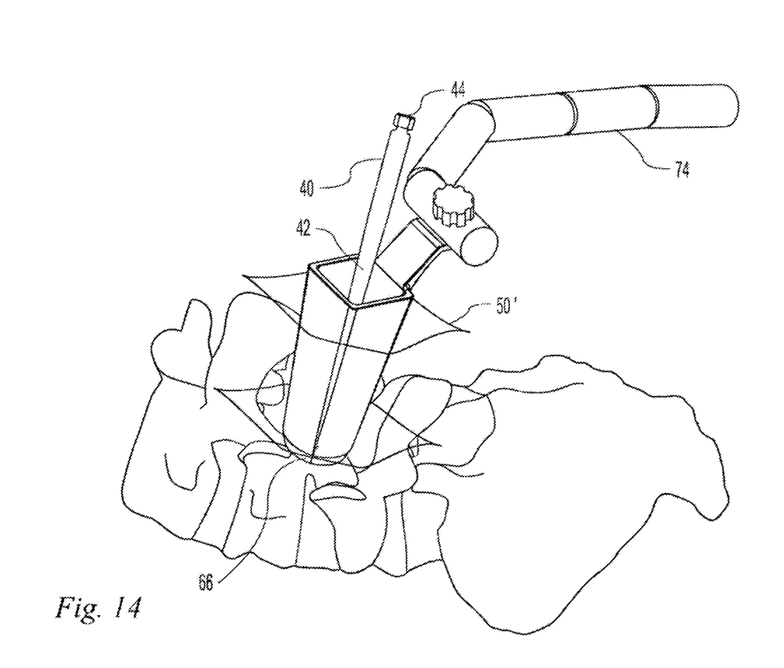

In another embodiment, a method of inserting a surgical retractor into an incision with the aid of a surgical retractor insertion tool is illustrated in FIGS. 11-14. The method may comprise making an incision 50 in a patient. The method also may comprise inserting a surgical retractor insertion tool 40 into incision 50 as shown in FIG. 11. Surgical retractor insertion tool 40 may be inserted into incision 50 in a direction parallel to incision 50. The method may further comprise rotating surgical retractor insertion tool 40 to a position no longer parallel with incision 50 as shown in FIG. 12. Such rotation may cause the edges of incision 50 to separate and form a widened incision 50′. The method may further comprise removing a handle 48 of surgical retractor insertion tool 40 from surgical retractor insertion tool 40 as shown in FIG. 13. The method may also comprise inserting a surgical retractor 10 into the widened incision 50′ as shown in FIG. 14. Surgical retractor 10 may be inserted into widened incision 50′ by passing surgical retractor 10 over surgical retractor insertion tool 40 inserted in incision 50 and rotated to create widened incision 50′.

Making an incision 50 in a patient may be a part of any type of surgery and may be accomplished according to any procedure well known to those of ordinary skill in the art. Exemplary surgeries which may entail making incision 50 may include, but are not limited to, spinal surgery or disc/pedicle surgery. Incision 50 may be made in any type of patient, including human or animal patients. In one aspect, incision 50 may be a generally straight line incision. Incision 50 may also be equal in length or longer than a width of surgical retractor insertion tool 40 so that incision 50 may accommodate the insertion of surgical retractor insertion tool 40 into incision 50.

The insertion of surgical retractor insertion tool 40 into incision 50 may be accomplished manually or by any other suitable means for inserting surgical retractor insertion tool 40 into incision 50. Surgical retractor insertion tool 40 may be any suitable surgical retractor insertion tool. In one aspect, surgical retractor insertion tool 40 may be as described in detail above. More specifically, surgical retractor insertion tool 40 may comprise a paddle-shaped spreader 42 having a first end 44 and a second end 46 opposite first end 44 and a handle 48 removably coupled to first end 44 of paddle-shaped spreader 42. In one aspect, surgical retractor insertion tool 40 may be inserted into incision 50 in a direction parallel to incision 50. In other words, the first and second broad faces of paddle-shaped spreader 42 may be aligned in a plane parallel with incision 50. The thickness of paddle-shaped spreader 42 may be approximately equal to incision 50 such that surgical retractor insertion tool 40 may be inserted into incision 50. Surgical retractor insertion tool 40 may be inserted into incision 50 at any suitable depth that will allow surgical retractor insertion tool 40 to separate the edges of incision 50 when surgical retractor insertion tool 40 is rotated.

The rotation of surgical retractor insertion tool 40 may be accomplished manually or by any other suitable means for rotating surgical retractor insertion tool 40 when inserted in incision 50. In one aspect, surgical retractor insertion tool 40 may be rotated by rotating handle 48 removably coupled to first end 44 of surgical retractor insertion tool 40. Surgical retractor insertion tool 40 may be rotated to any position not in parallel with incision 50. In one aspect, surgical retractor insertion tool 40 may be rotated 90 degrees to a position perpendicular to incision 50 to facilitate separation of the edges of incision 50 to create widened incision 50′.

After rotating surgical retractor insertion tool 40, handle 48 may be removed from surgical retractor insertion tool 40. Removal of handle 48 may be accomplished manually or by any other suitable means for removing handle 48 from surgical retractor insertion tool 40. Removal of handle 48 may be dictated by the manner in which handle 48 is removably coupled to surgical retractor insertion tool 40. For example, where handle 48 is removably coupled to surgical retractor insertion tool 40 by screwing handle 48 onto first end 44, handle 48 may be removed by unscrewing handle 48 from first end 44.

Once handle 48 has been removed, surgical retractor 10 may be inserted into widened incision 50′. Surgical retractor 10 may be any suitable surgical retractor. In one aspect, surgical retractor 10 is a surgical retractor as described in detail above. Surgical retractor 10 may be inserted into widened incision 50′ by sliding surgical retractor 10 down surgical retractor insertion tool 40 and into widened incision 50′. In other words, surgical retractor insertion tool 40 is passed through opening 18 of surgical retractor 10. Surgical retractor 10 may be passed over surgical retractor insertion tool 40 manually or by any other means for passing surgical retractor 10 over surgical retractor insertion tool 40 and into widened incision 50′. Surgical retractor 10 may have a size that is matched to widened incision 50′. In other words, surgical retractor 10 may have a size that will allow surgical retractor 10 to be inserted into widened incision 50. Surgical retractor 10 may have a chamfer 66 to aid in introducing second insertion end 16 into the widened incision 50′. Similarly, opening 18 of surgical retractor may have a size approximately equal to or greater than surgical retractor insertion tool 40 such that surgical retractor 10 may be passed over surgical retractor insertion tool 40. The surgical retractor 10 may then be attached to a stabilizing device such as flexible arm 74.

Once surgical retractor 10 is inserted into widened incision 50′, the edges of widened incision 50′ may be held apart by surgical retractor 10. Accordingly, the method may comprise removing surgical retractor insertion tool 40 from widened incision 50′ after surgical retractor 10 has been inserted in widened incision 50′. Alternatively, surgical retractor insertion tool 40 may remain in place for a portion or for the duration of the surgery.

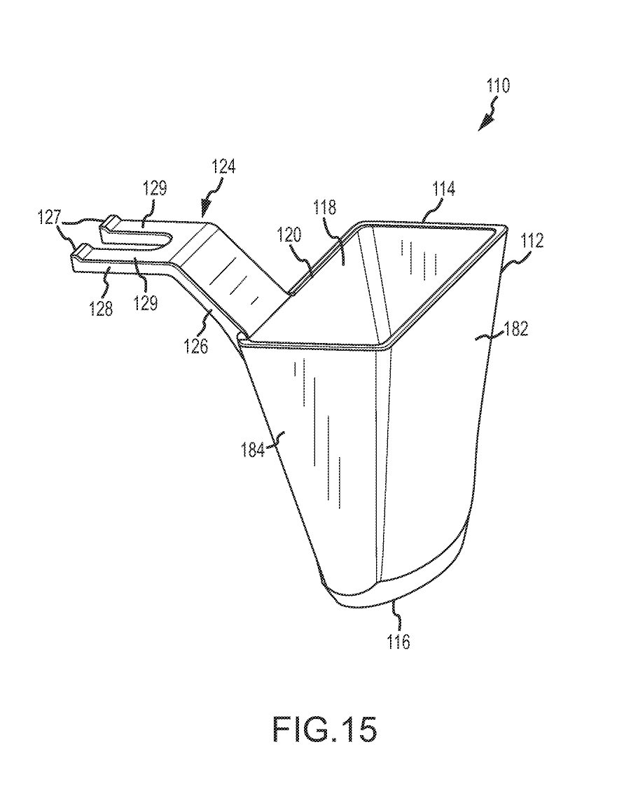

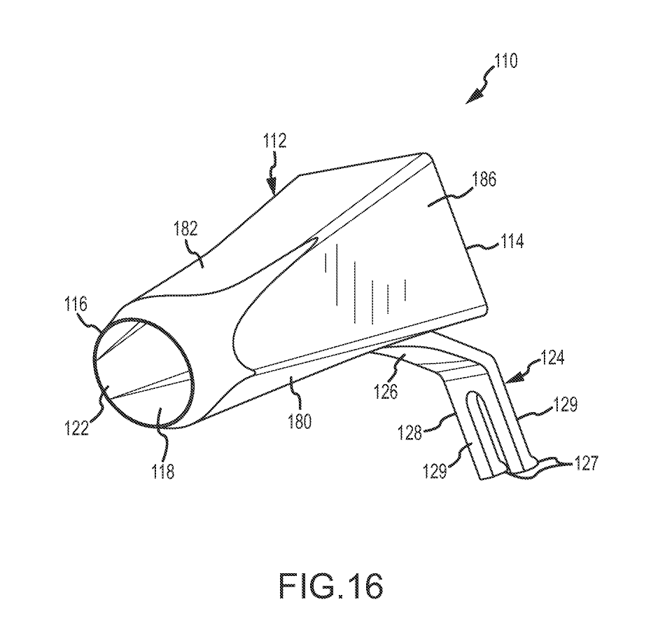

As shown in FIGS. 15-22, another example surgical retractor 110 is shown and described. The surgical retractor 110 may include an asymmetrical feature, such as a lateral or radial offset between openings at opposing ends of the retractor body that provides asymmetry. The surgical retractor 110 may be referred to as an asymmetrical surgical retractor. The surgical retractor 110 may have openings at opposing ends of the retractor body that have different sizes and shapes. In one example, an insertion end of the surgical retractor 110 has a curved opening (e.g., an elliptical opening), and a protrusion end of the surgical retractor 110 has a polygonal opening (e.g., rectangular opening).

The relatively elongate opening shapes of at least the insertion end of the surgical retractor may help minimize tissue (e.g., muscle) disruption when the surgical retractor is inserted into a patient. The elongate opening shapes of the openings at the protrusion and insertion ends also may improve visualization of internal tissue when the surgical retractor is inserted through the incision. For example, with a lateral offset of the openings, the generally elongate construction of the openings, and/or an increased size of the opening at the protrusion end compared to the opening size at the insertion end may provide an increased range of insertion angles for tool and other devices inserted through the surgical retractor 110 into the patient.

In some embodiments, the surgical retractor 110 may generally comprise a retractor body 112 having a first protrusion end 114 and a second insertion end 116 opposite first protrusion end 114. Surgical retractor 110 may also comprise an opening 118 (also referred to herein as a pass through aperture) that extends through the entire length of retractor body 112 (i.e., from first protrusion end 114 to second insertion end 116). Because opening 118 extends through the entire length of the retractor body 112, opening 118 may comprise a first end opening 120 located at first protrusion end 114 of retractor body 112 and a second end opening 122 located at second insertion end 116 of retractor body 112. First end opening 120 may be larger than second end opening 122 such that opening 118 is defined by a generally conical or tapering shape that tapers from first end opening 120 to second end opening 122. The shape and size possibilities for the first and second end openings 120, 122 may be similar to those described above related to first and second end openings 20, 22. The first and second end openings 120, 122 may be arranged in planes that are parallel and spaced apart longitudinally.

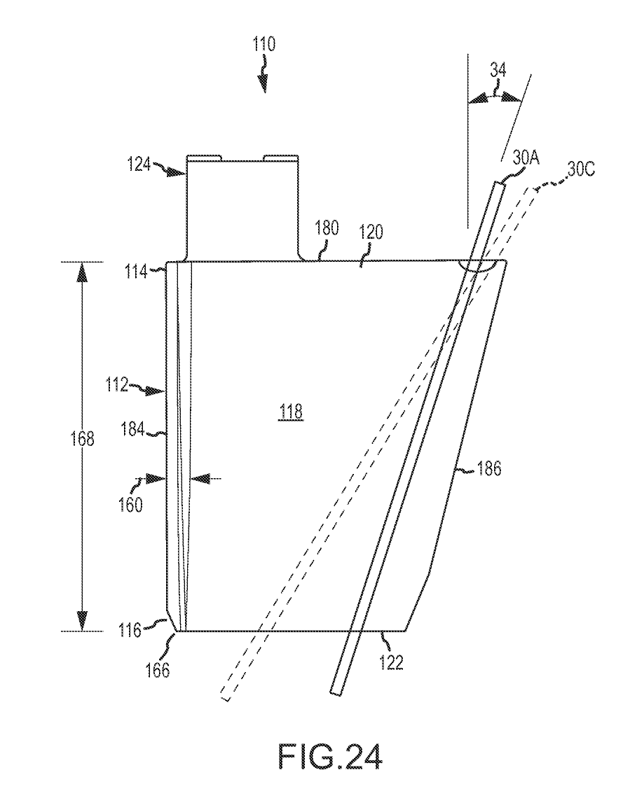

In this embodiment the retractor body 112 has a generally constant and relatively thin wall thickness 160 such that the retractor body 112 mimics the shape of the opening 118 (see FIG. 24). The retractor body 112 may have a chamfer 166 at the second insertion end 116. The chamfer 166 may effectively make the size of the second end opening 122 the size the retractor body 112 at the second insertion end 116. The retractor body 112 may have a height 168 (see FIG. 24) between the first protrusion end 114 and second insertion end 116. In one example, the height 168 may be in the range of about 20 mm to about 100 mm, and more preferably in the range of about 35 mm to about 65 mm.

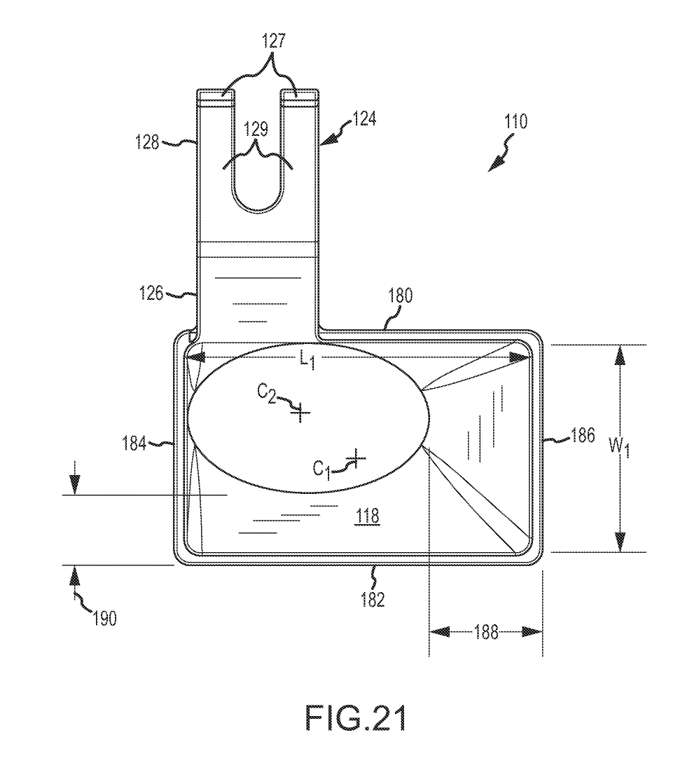

As shown in FIG. 21, in some embodiments the first end opening 120 has a first length L₁, a first width W₁, and a first central axis C₁. The first length L₁ may be greater than the first width W₁. The length L₁ may be in the range of about 20 mm to about 40 mm, and more preferably in the range of about 30 mm to about 35 mm. The width W₁ may be in the range of about 10 mm to about 30 mm, and more preferably in the range of about 15 mm to about 25 mm.

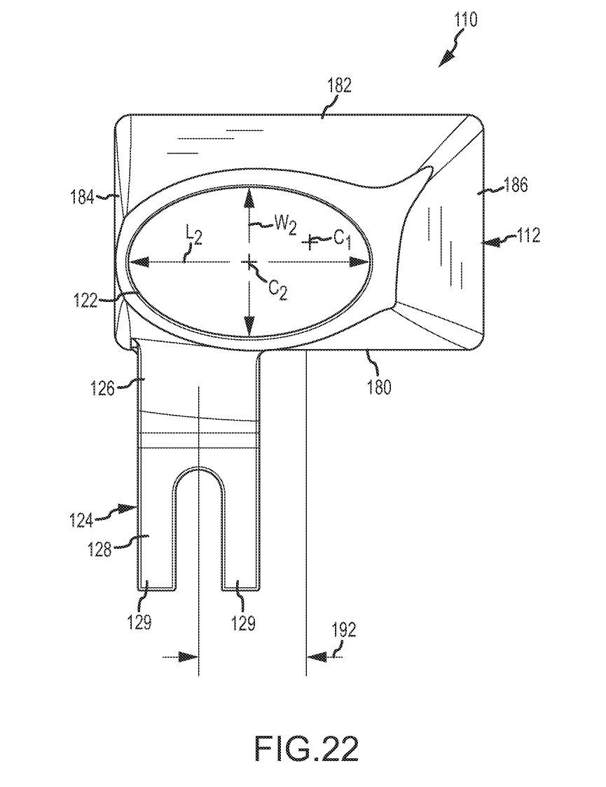

As shown in FIG. 22, the second end opening 122 has a second length L₂, a second width W₂, and a second central axis C₂. The second length L₂ may be greater than the second width W₂. The length L₂ may be in the range of about 15 mm to about 35 mm, and more preferably in the range of about 20 mm to about 30 mm. The width W₂ may be in the range of about 5 mm to about 25 mm, and more preferably in the range of about 10 mm to about 20 mm. Each of the first and second end openings 120, 122 may have a major axis dimension and a minor axis dimension aligned with the length and width dimensions, respectively. Other examples are possible that include different ranges of sizes for the first and second end openings 120, 122. Further, in some embodiments the first and/or second end openings 120, 122 may be generally square, with the corresponding width about equal to the length of the opening.

The retractor body 112 may have a generally conical or tapered shape that tapers from the first length L₁ and first width W₁ to the second length L₂ and second width W₂, respectively. In some embodiments, the opening 118 may define one shape while the retractor body 112 defines another, different shape. In the example of FIGS. 15-22, the opening 118 defines a shape that is substantially similar to a shape of the retractor body 112.

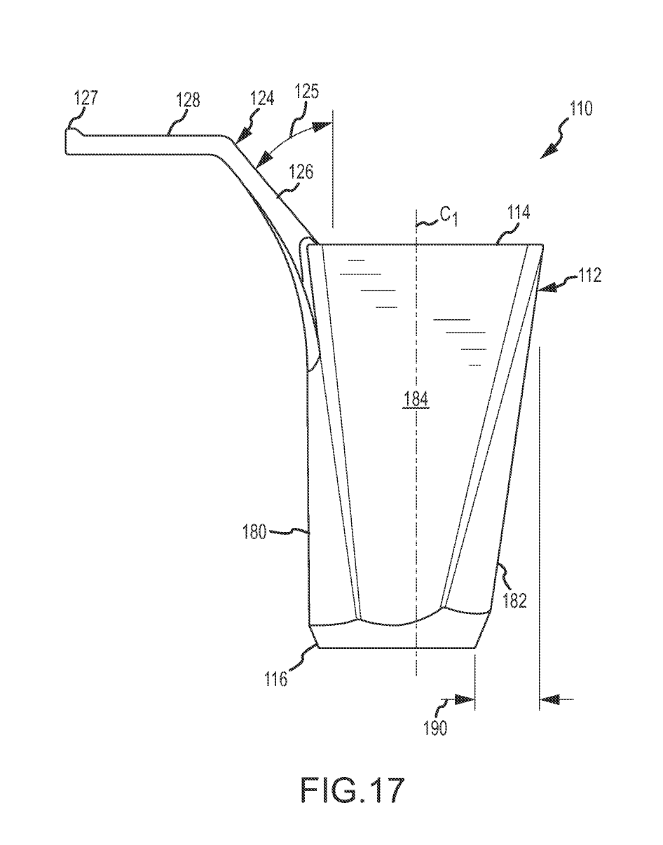

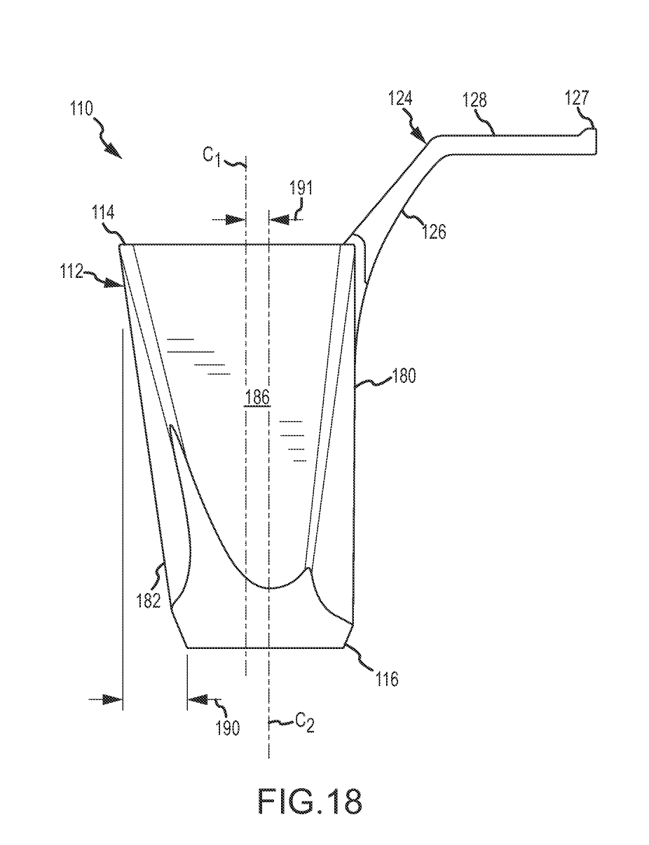

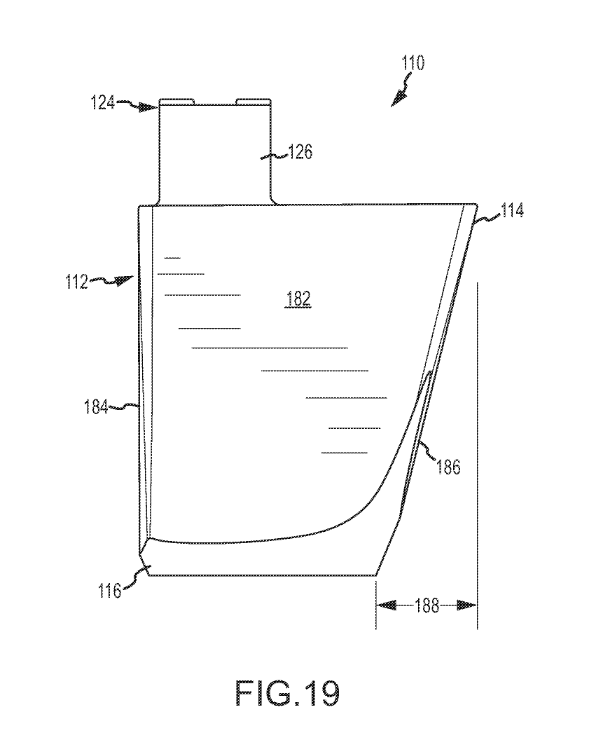

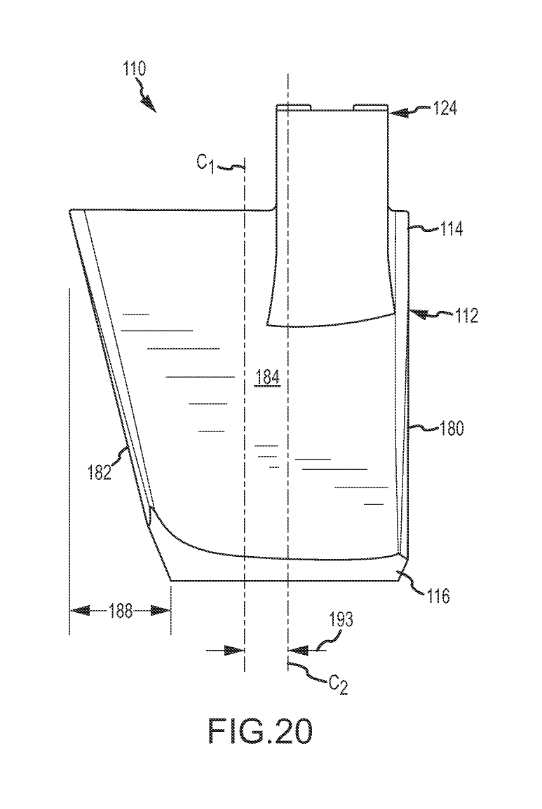

The retractor body 112 may have a plurality of side surfaces, including a lateral side 180, a medial side 182, a cephalad side 184, and a caudad side 186. The first and second end openings 120, 122 may be arranged with the first and second central axes C₁, C₂ coaxial so that each of the sides 180, 182, 184, 186 are angled relative to the axes C₁, C₂. In other examples, the first and second central axes C₁, C₂, are arranged offset laterally (i.e., radially) from each other. The first and second central axes C₁, C₂ may be offset sufficiently to arrange at least one of the sides 180, 182, 184, 186 in parallel with one of the central axes C₁, C₂. Alternatively, first and second central axes C₁, C₂ may be offset sufficiently to position at least one of the sides 180, 182, 184, 186 at a different angle with one of the central axes C₁, C₂ than the other sides have with that axis or axes. FIGS. 17 and 18 illustrate the lateral side 180 arranged in parallel with first central axis C₁ and medial side 182 arranged at an angle that positions the second insertion end 116 at a medial offset 190 from the first protrusion end 114. FIGS. 19 and 20 illustrate the cephalad side 184 arranged in parallel with the first central axis C₁ and caudad side 186 arranged at an angle that positions the second insertion end 116 at an inferior offset 188 from the first protrusion end 114.

The first and second central axes C₁, C₂ may be offset from each other a first lateral offset distance 191 (see FIG. 18) in a direction between the cephalad and caudad sides 184, 186. The first and second central axes C₁, C₂ may be offset from each other a second lateral offset distance 193 (see FIG. 20) in a direction between the lateral and medial sides 180, 182. The lateral offset(s) between the first and second end openings 120, 122 may help minimize tissue retraction. The lateral offset(s) between the first and second end openings 120, 122 may maximize room to provide a proper trajectory for inserting tools, implants, fasteners (e.g., screws), etc. through the surgical retractor and into the patient. For example, the construction of surgical retractor 110 may provide desired instrument clearance in the medial and caudad direction when an implant, such as a pedicle screw, an interbody spacer, or the like, is angled in the lateral and cephalad direction. Other orientations for the lateral offset of the first and second end openings 120, 122 relative to the sides 180, 182, 184, 186 may be advantageous for other implant trajectories. In addition, in some embodiments the first and second openings 120, 122 may have both a first lateral offset 191 and a second lateral offset 193.

In application, surgical retractor 110, as shown in FIGS. 15-22, may be inserted into a wound or incision made in a patient to open up and hold open the wound or incision area. The elongate cross-sectional shape of the surgical retractor 110 (as defined by lengths L₁, L₂ being greater than widths W₁, W₂, respectively) may provide improved access to and visibility of the wound or incision area.

Surgical retractor 110 may be designed such that second insertion end 116 serves as the insertion end and first protrusion end 114 serves as the portion of the surgical retractor that protrudes out of the patient. Retractor body 112 keeps the edges of the wound or incision spread apart to allow for access into the wound or incision area. That is to say, upon insertion, the edges of the wound or incision rest against the exterior of retractor body 112 and are retained apart by the retractor body 112. Once in place, opening 118, including first end opening 120 and second end opening 122, provides the pathway for viewing inside the wound or incision as well as for inserting surgical tools into the wound or incision. Such surgical tools may generally be introduced into the wound or incision by passing the tools through first end opening 120 and down opening 118 to second end opening 122.

As best seen in FIGS. 15, 16, 21 and 22, first end opening 120 and second end opening 122 may have specific shapes designed to improve the usefulness of surgical retractor 110. Specifically, first end opening 120 may have a polygon shape and second end opening 122 may have a curved shape. While FIGS. 15, 16, 21 and 22 illustrate a rectangular-shaped first end opening 120 and an elliptical shaped second end opening 122, any type of polygon or curve, or combination of polygon and curved features, may be used.

While, polygon shapes are described above for first end opening 120, the shape of first end opening 120 is not limited. First end opening 120 may have a circular shape or a shape utilizing both straight segments and curved segments. For example, in one embodiment first end opening 120 has a generally rectangular shape, with one or more of the corners curved more than the other corners. For example, first end opening 120 may have three, two or one generally right-angle corners, with one, two or three curved corners, respectively. Likewise, while curved shapes are described for second end opening 122, the shape of second end opening is not limited. Second end opening 122 may have a polygon shape or a shape utilizing both straight segments and curved segments.

When first end opening 120 comprises a polygon shape, surgical retractor 110 may include corners in the proximity of first protrusion end 114. For example, as shown in FIG. 21, the polygonal shape of first end opening 120 provides multiple corners at first protrusion end 114 of surgical retractor 110. Where second end opening 122 has a circular or other curved shape, these corners will gradually transition to the circular or other curved shape near second insertion end 116 (as shown in e.g., FIGS. 15-22), but corners will remain at first protrusion end 114 of surgical retractor 110.

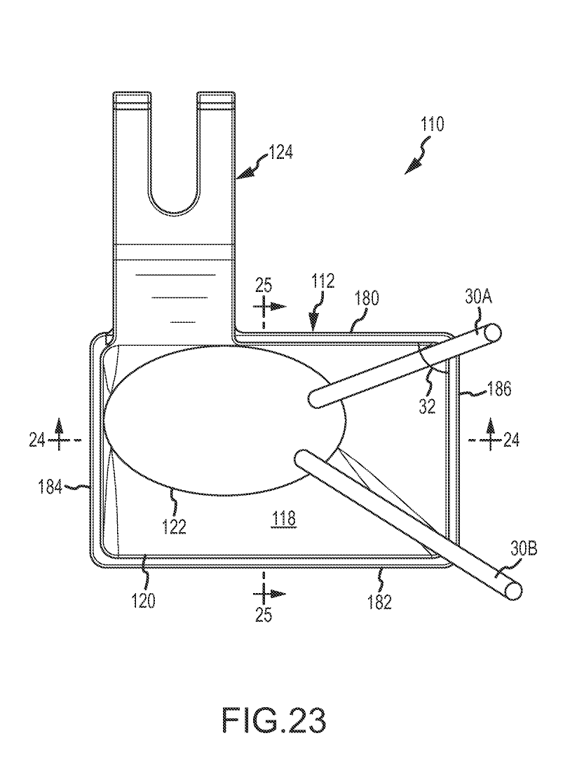

As shown in FIGS. 23-25, such corners may be used for resting surgical tools 30 not in use or which need to be held in a certain position while other tools are being used (such as, e.g., a light). The tools will tend to settle or be constrained in the corners such that the corners will hold tools 30 in place, unlike rounded edges which would allow tools 30 to move freely along the curved sides. To further maintain tools 30 in place, the corners may include a tool retention feature such as tool retention loop 32 near first protrusion end 114 of surgical retractor 110. Tool retention loops 32 may generally comprise loops that extend across a corner and allow tools 30 to be inserted through the loop. The size of tool retention loops 32, including how much room is provided within the loop, is not limited. Tool retention loops 32 may be large enough to accept a variety of tools having different sizes. In one aspect, tool retention loops 32 may be adjustable. For example, tool retention loops 32 may be tightened to better secure a smaller tool, or may be enlarged to accommodate a larger tool. Any number of tool retention loops 32 may be provided, including a single tool retention loop 32 or a tool retention loop 32 for every corner of the polygon-shaped first end opening 120. The tool retention loop 32 may open radially in the form of a clip that allows the tool to engage the tool retention loop 32 radially in, for example, a snap-fitting relationship.

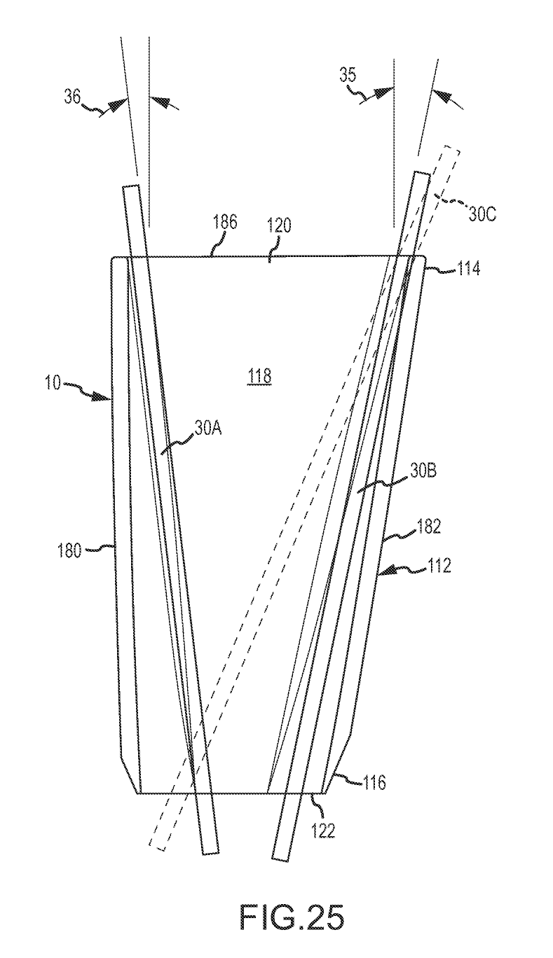

The generally elongate shape of the surgical retractor 110 (i.e., greater length L₁, L₂ than widths W₁, W₂, respectively), permits an increased angle of insertion of the tool 30 relative to the central axes C₁, C₂. FIGS. 24 and 25 illustrate various insertion angles 34, 35, 36 for the tool permitted by the elongate shape of first and second end openings 120, 122. The angles 34-36 may be further enhanced by the inferior and medial offsets 188, 190. The tool 30 is shown in broken line in FIGS. 24 and 25 to illustrate a range of angled positions possible for the tool 30 extending through the surgical retractor 110. The angles 34-36 for tool 30 also represent an insertion angle for other tools, instruments, and devices such as, for example, a pedicle screw.

The features of a generally elongate construction of the first and second end openings 120, 122, the use of inferior and/or medial offsets 188, 190, and providing the first end opening 120 of greater size than the second end opening 122, alone or in some combination, may provide the benefit of increased insertion angles for tools and devices through the surgical retractor 110 and into the patient.

Surgical retractor 110 may further comprise a mounting bracket 124. Mounting bracket 124 may be used to secure surgical retractor 110 to a frame or to other equipment used when surgical retractor 110 is in place in a wound or incision. Mounting bracket 124 may generally be coupled to retractor body 112 at any location along retractor body 112. In one aspect, mounting bracket 124 is coupled to retractor body 112 at a location proximate first protrusion end 114 of retractor body 112. Mounting bracket 124 may generally protrude away from retractor body 112 and also may protrude away from second insertion end 116 of retractor body 112. Mounting bracket 124 may be coupled to retractor body 112 using any suitable mechanism, such as glue, welding, bolts, or screws. In one aspect, mounting bracket 124 may be integrally formed with retractor body 112 such that surgical retractor 110 is one unitary piece. In one embodiment, mounting bracket 124 is coupled to or formed with retractor body 112 at a location which provides a desired mounting functionality for retractor 112 without physically or visually interfering with tools inserted down opening 118. In some embodiments, bracket 124 is mounted on or near first protrusion end 114 at a desired position relative to offsets 188, 190. For example, bracket 124 may be mounted near an angled side 180, 182, 184, 186 or near a straight side 180, 182, 184, 186. In this manner, mounting bracket may be positioned closer to or further from second end opening 122 as desired.

When first end opening 120 of surgical retractor 110 has a polygon shape, mounting bracket 124 may be coupled to surgical retractor 10 centrally along a side of retractor body 112 formed by the polygon shape of first end opening 120, for example. Alternatively, mounting bracket 124 may be positioned offset from a central location along a side of the polygon shape of first end opening 120 (as shown in FIGS. 15-22) a bracket offset 192 (see FIG. 22). Providing a bracket offset 192 for the mounting bracket 124 may limit interference of the mounting bracket 124 with a trajectory of a driver, tool or other device that is inserted into the opening 118 of the surgical retractor 110.

For example, for some embodiments the polygon shape of first end opening 120 is a rectangle. Mounting bracket 124 may be coupled to any one of the four sides of the rectangle shape or any one of the corners. Where the polygon shape of first end opening 120 has between three and eight sides, mounting bracket 124 may be coupled to retractor body 112 on any of the three to eight sides or the associated corners between the sides.

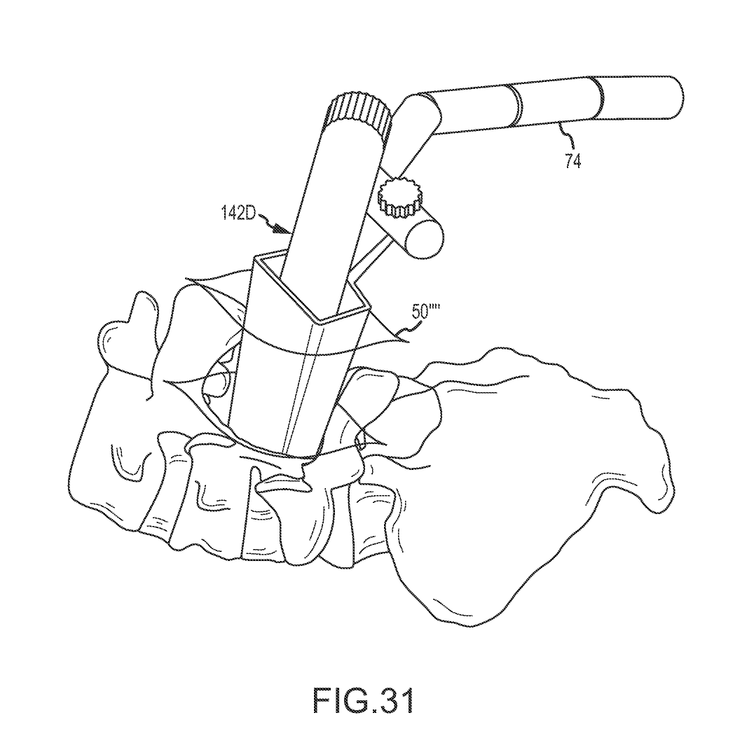

The shape of mounting bracket 124 may be any suitable shape for allowing mounting bracket 124 to be secured to a frame or other equipment used when surgical retractor 110 is in place. As shown in FIGS. 15-22, mounting bracket 124 may generally comprise an angled section 126 coupled to retractor body 112. Angled section 126 may generally protrude away from both second insertion end 116 and retractor body 112 of surgical retractor 110. Angled section 126 may generally be at an angle 125 (see FIG. 17) of about 45 degrees relative to an axis running through opening 118 of surgical retractor 110 (e.g., first central axis C₁), although other angles may also be used. Mounting bracket 124 may further comprise a transverse section 128 that is coupled directly to angled section 126. Transverse section 128 may generally be transverse to the axis running through opening 118 of surgical retractor 110. As shown in FIG. 15, transverse section 128 may comprise two arms 129 with a slot located therebetween. The slot may serve as the area where surgical retractor 110 may be secured to a frame or other equipment (e.g., see equipment 74 in FIG. 31) used in conjunction with surgical retractor 110. One or both arms 129 may also include a rib or boss 127 at the end of arms 129.

The dimensions of surgical retractor 110 are not limited and may be adjusted depending on the size of the wound or incision with which surgical retractor 110 is being used. As noted above, one limitation on dimensions may be that first end opening 120 is larger than second end opening 122. A first end opening 120 that is larger than a second end opening 122 may generally mean that retractor body 112 tapers inwardly between first end opening 120 and second end opening 122. The material of surgical retractor 110 is also not limited. In one aspect, surgical retractor 110 may comprise biocompatible material. In a particular embodiment, retractor 110 comprises a plastic or other material that is radiolucent. Different parts of surgical retractor 110 may be made of the same or different materials. For example, mounting bracket 124 may be the same or a different material than the material of retractor body 112.

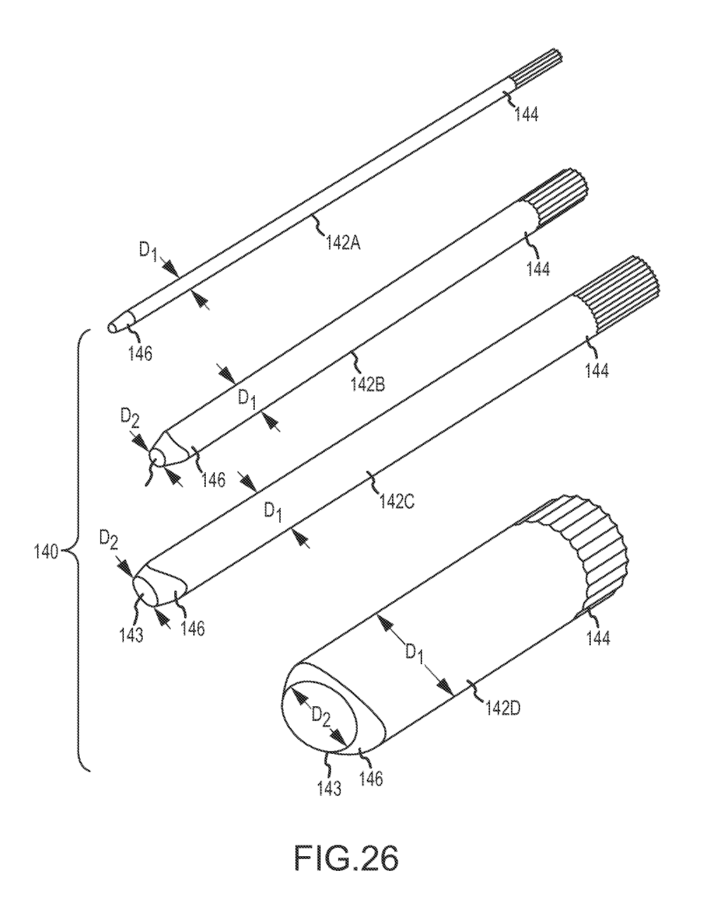

A surgical retractor insertion tool, such as tool 40 described above with reference to FIGS. 9-14, may be used to place surgical retractor 110 in a wound or incision is disclosed. FIGS. 26-31 illustrate a surgical retractor insertion tool assembly 140 that includes a plurality of dilators 142A-D of different cross-sectional size. Each of dilators 142A-D may include first and second ends 144, 146 and an outer diameter D₁. The dilators 142A-D may further include an opening or pass through aperture 143 having an inner diameter D₂. The outer diameter D₁ of each dilator 142A-D may be sized smaller than the inner diameter D₂ of the opening 143 of the next larger diameter dilator 142A-D.

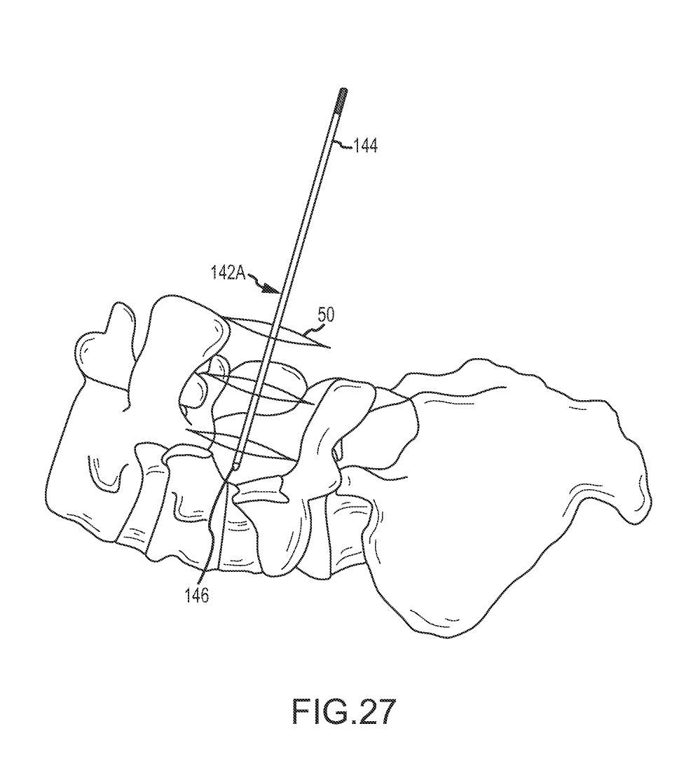

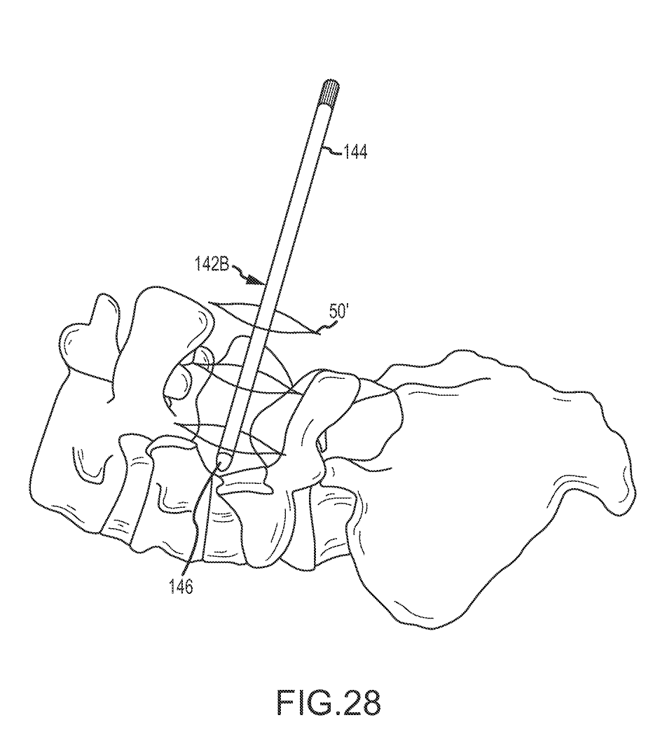

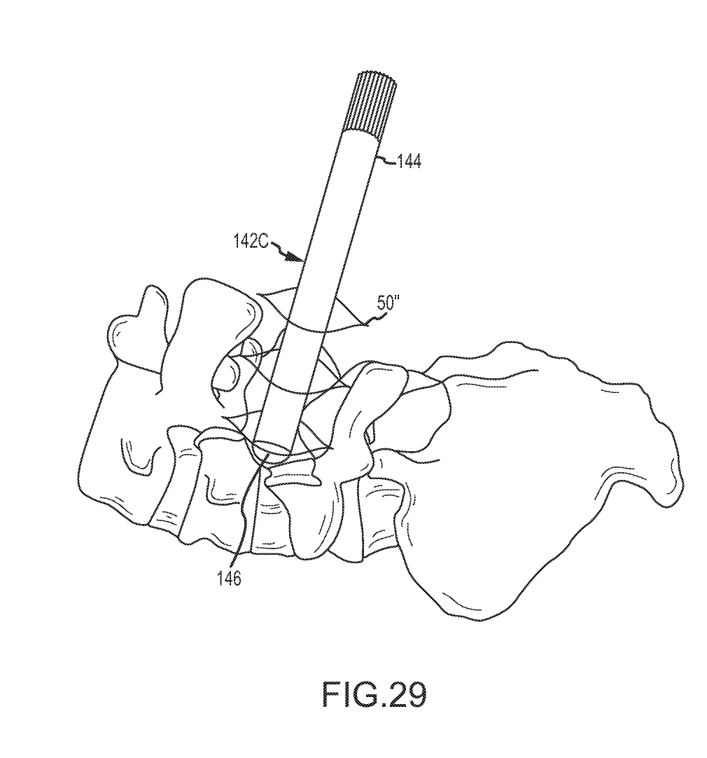

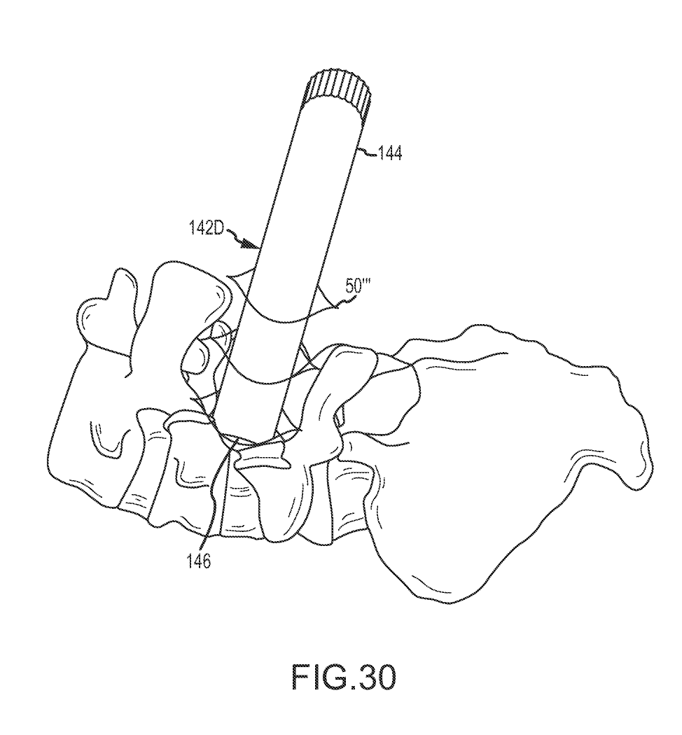

In operation, the dilator 142A may be inserted into an incision 50 as shown in FIG. 27. The dilator 142A has an outer diameter D₁ that is less than the inner diameter D₂ of the dilator 142B so that the dilator 142B can extend over the dilator 142A while the dilator 142A is positioned in the incision 50 to further dilate to create dilated incision 50′ as shown in FIG. 28. The dilator 142C is extended over dilator 142B to create further dilated incision 50″ as shown in FIG. 29. The dilator 142D is extended over dilator 142C to create further dilated incision 50′″ as shown in FIG. 30. Any number of dilators 142 may be included in the surgical retractor insertion tool assembly 140 and used in any order to dilate the incision 50 to a desired size.

In addition, dilators 142 may have a generally circular cross-sectional shape or they may have an elliptical or other curved shape. In one embodiment, the innermost dilator (e.g., dilator 142A) has a generally circular cross-section, and outermost dilator (e.g., dilator 142D) has a generally elliptical cross-section. Dilators 142 between the innermost and outermost dilators (e.g., dilators 142B and 142C) may have cross-sectional shapes that transition from circular to elliptical. In a particular embodiment, dilator(s) 142 have a shape or outer diameter D₁ that generally corresponds with the shape of second end opening 122.

Upon reaching a desired dilated size for incision 50, the surgical retractor 110 is extended over the dilators 142 positioned in the incision and to create yet further dilated incision 50″″, as shown in FIG. 31. The operator may then remove the dilators 142A-D and prepare the surgical retractor 110 to receive a tool 30 or other device through the opening 118 to treat the patient within the incision.

Typically, the dilators 142A-D of the surgical retractor insertion tool assembly 140 are arranged generally parallel with the wound or incision. Ideally, the dilators 142A-D are inserted between muscle fibers (not specifically shown). Successive insertion of increasing larger dilators 142A-D into the wound or incision may spread apart the edges of the wound or incision, thereby making it easier to insert a surgical retractor.

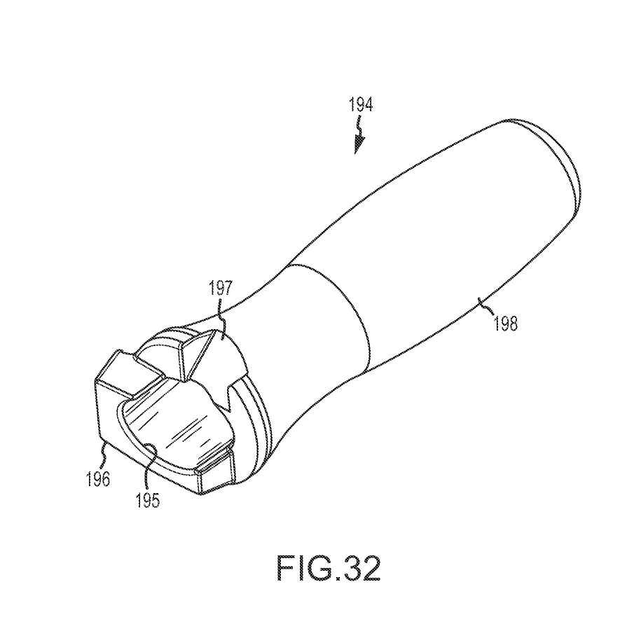

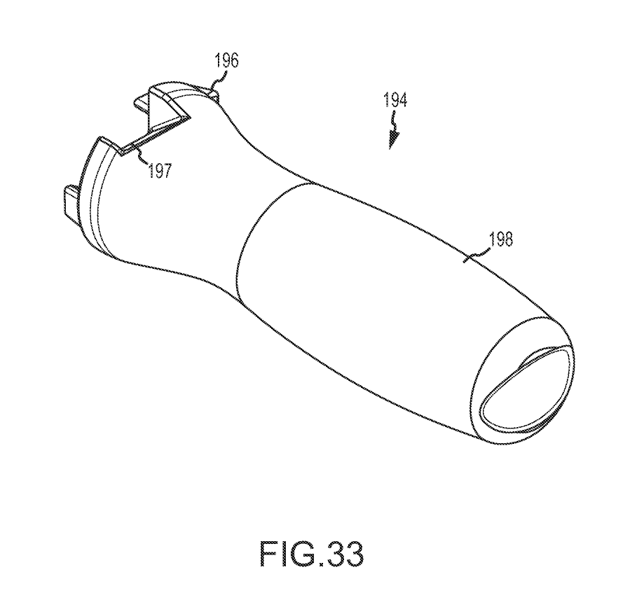

The surgical retractor 110 may be carried and/or positioned relative to an incision by a placement tool 194 (see FIGS. 32-34). The placement tool 194 may include a handle 198 at one end, and a surgical tool interface at an opposite end. In some embodiments, the surgical tool interface includes a polygonal portion 196 that interfaces with the first end opening 120, and a bracket portion 197 that interfaces with the mounting bracket 124. In one embodiment, bracket portion 197 is angled to generally match the angle of angled section 126 of mounting bracket 124. The placement tool 194 may be connected to, coupled with, or otherwise engage the surgical retractor 110 in any manner such as, for example, an interference fit, a snap-fit, or a releasable fastener. In some embodiments, polygonal portion 196 is sized and shaped to interface with first end opening 120. In some embodiments, placement tool 194 may have a cavity or lumen 199 running the length of handle 198. In this manner, the cavity 199 can provide a viewport down through tool 194 and into retractor 110, such as when tool 194 and retractor 110 are coupled together. This may be useful, for example, in aligning retractor 110 with an incision, or with other instruments such as dilation tubes 142 over which retractor 110 may be inserted. If desired, an additional instrument, such as paddle 42, can be inserted down the lumen in placement tool 194. In some embodiments, placement tool 194 further includes a curved portion 195 that mirrors a shape and size of the second end opening 122. Curved portion 195 may define the cavity or lumen 199 through placement tool 194. Many shapes, sizes and general constructions are possible for the placement tool 194.

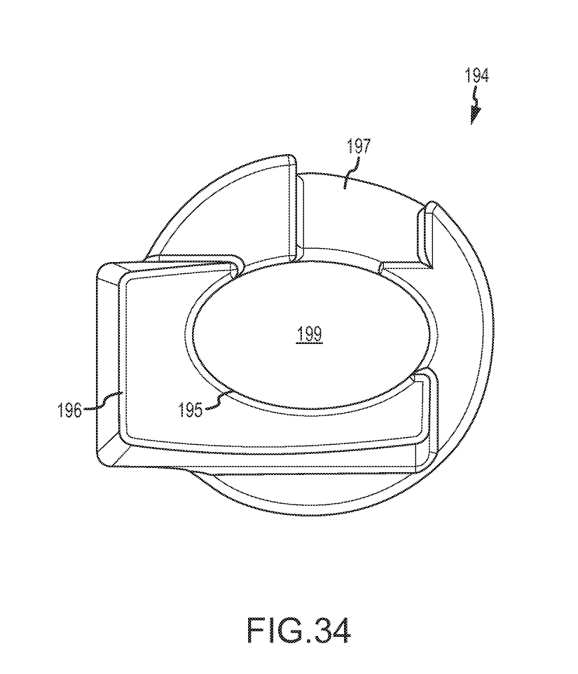

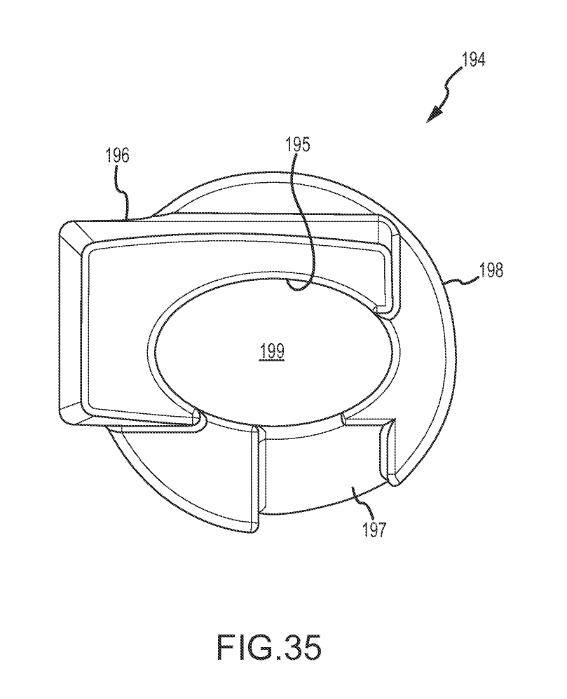

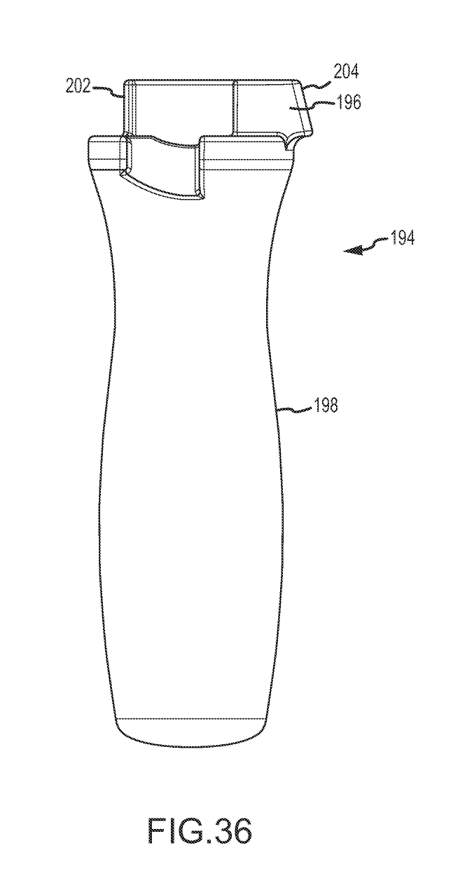

In some embodiments, polygonal portion 196 is positioned offset relative to curved portion 195. For example, with reference to the end view of FIG. 34, curved portion 195 is in the upper right quadrant of polygonal portion 196. Alternatively, with reference to the end view of FIG. 35, curved portion 195 is in the upper left quadrant of polygonal portion 196. Other embodiments use alternative alignments of curved portion 195 and polygonal portion 196 to engage retractor 110. In these embodiments, polygonal portion 196 is adapted to fit first end opening 120, while curved portion 195 and lumen 199 are aligned with second end opening 122. Polygonal portion 196 may further include sides having different lengths and/or orientations. For example, as shown in FIG. 36, polygonal portion 196 has at least one straight or generally straight side portion 202 and at least one angled side portion 204. In this manner, angled side portion 204 may be angled an amount to generally correspond with an angle of one or more sides 180, 182, 184, 186 of body 112. For example, angled side portion 204 may correspond to side 186 in FIG. 23, while side portion 202 corresponds to side 184.

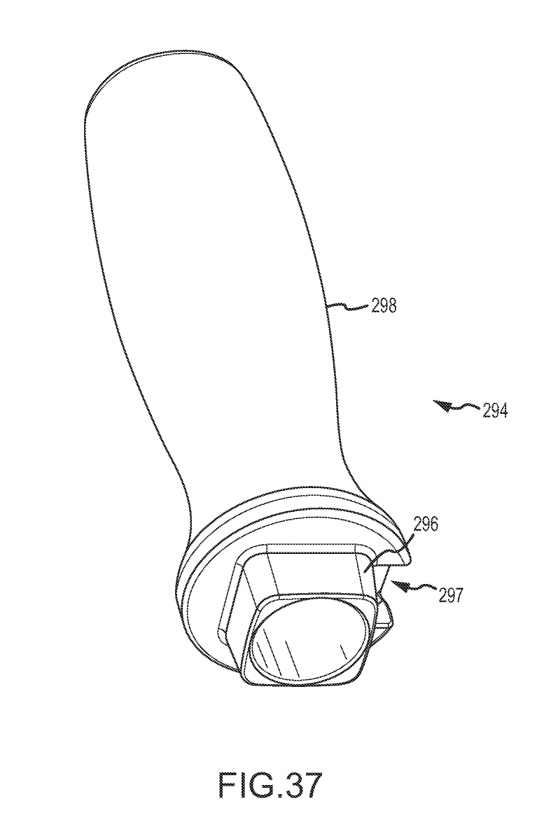

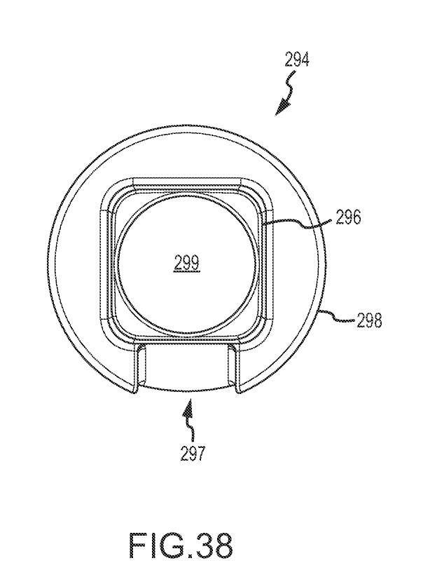

Referring now to FIGS. 37-38, an alternative tool 294 may include a handle 298 at one end, and a surgical tool interface at an opposite end. In some embodiments, the surgical tool interface includes a polygonal portion 296 that interfaces with the first end opening 120, and a bracket portion 297 that interfaces with the mounting bracket 124. A lumen 299 may run through handle 298 to allow tool 294 to be inserted over one or more dilators 142. In this embodiment, polygonal portion 296 is generally square or rectangular, to match with a generally square or rectangular first end opening 120, such as that depicted in FIG. 3.

In another aspect, a method for inserting a surgical retractor into an incision made in a patient is disclosed. The surgical retractor 110 may simply be inserted directly into an incision by pressing second insertion end 116 into the incision. The second insertion end 116 may include a chamfer 166 to aid the insertion. The elongate construction of the second end opening 122 may assist in easier insertion of the second insertion end 116 into the incision, and may minimize tissue damage in the area of the incision.

In another embodiment, a method of inserting a surgical retractor into an incision with the aid of a surgical retractor insertion tool assembly is illustrated in FIGS. 26-31 as explained above. The method may comprise making an incision 50 in a patient. The method may also comprise inserting a first dilator 142A of the surgical retractor insertion tool assembly 140 into incision 50 as shown in FIG. 26. The method may further comprise inserting a second dilator 142B over the first dilator 142A to provide an increased size incision 50′, as shown in FIG. 27. The method may further comprise inserting a third dilator 142C over the second dilator 142B to provide a further increased size incision 50″, as shown in FIG. 28. The method may also comprise inserting a fourth dilator 142D over the third dilator 142C to provide a still further increased size incision 50′″, as shown in FIG. 29. Surgical retractor 110 may be inserted over the fourth dilator 142D of the surgical retractor insertion tool assembly 140 by passing surgical retractor 110 over fourth dilator 142D to provide a further increased size incision 50″″.

The insertion of surgical retractor insertion tool assembly 140 into incision 50 may be accomplished manually or by any other suitable means for inserting surgical retractor insertion tool assembly 140 into incision 50. Surgical retractor insertion tool assembly 140 may be any suitable surgical retractor insertion tool (e.g., the paddle shaped spreader 42 or the set of dilators 142A-D). In one aspect, surgical retractor insertion tool assembly 140 may be inserted into incision 50 in a direction parallel with a depth direction of incision 50. In other words, each of the dilators 142A-D may be aligned in a plane parallel with incision 50. The surgical retractor insertion tool assembly 140 may be inserted into incision 50 at any suitable depth that will allow surgical retractor insertion tool assembly 140 to separate the edges of incision 50 when each dilator 142A-D is inserted and then removed from incision 50.

The dilators 142A-D may be handled manually or by any other suitable means for carrying, inserting, and manipulating the dilators 142A-D when inserted in incision 50.

Surgical retractor 110 may be inserted into widened incision 50′″ by sliding surgical retractor 110 down any one of dilators 142A-D of surgical retractor insertion tool assembly 140 and into incision 50. In other words, surgical retractor insertion tool assembly 140 is passed through opening 118 of surgical retractor 110. Surgical retractor 110 may be passed over surgical retractor insertion tool assembly 140 manually or by any other means for passing surgical retractor 110 over surgical retractor insertion tool assembly 140 and into incision 50.

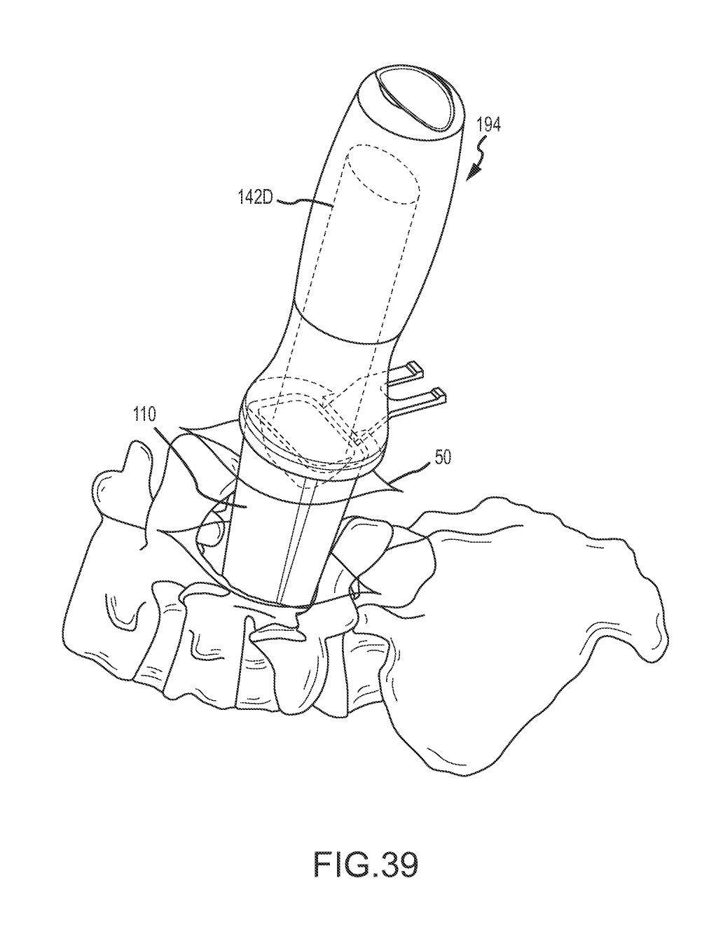

In some embodiments, and as shown in FIG. 39, retractor 110 is inserted into incision 50 with the assistance of tool 194 or 294. Retractor 110 may be inserted into incision 50 directly or into incision 50 over dilators 142. Tool 194 or 294 may then be engaged with retractor 110 as described in conjunction with FIGS. 32-38. For example, tool 194 may be guided over dilator(s) 142 such that polygonal portion 196 engages first end opening 120 of retractor 110. Retractor 110 may then be further advanced into incision 50 by pushing handle 198 towards incision 50. In this manner, tool 194 helps transfer force to retractor 110 in a controlled manner, while handle 198 provides a useful and comfortable interface for the user.

Surgical retractor 110 may have a size that is matched to any one of incisions 50, 50′, 50″, 50′″. In other words, surgical retractor 110 may have a size that will allow surgical retractor 110 to be inserted into dilated incision 50. Surgical retractor 110 may have a chamfer 166 to aid in introducing second insertion end 116 into the incision 50. Similarly, opening 118 of surgical retractor may have a size approximately equal to or greater than any one of the dilators 142A-D of surgical retractor insertion tool assembly 140 (or any other surgical retractor insertion tool that is used) such that surgical retractor 110 may be passed over surgical retractor insertion tool assembly 140. The surgical retractor 110 may then be attached to a stabilizing device such as flexible arm 74 (see FIG. 31).

Once surgical retractor 110 is inserted into incision 50, the edges of incision 50 may be held apart by surgical retractor 110. Accordingly, the method may further comprise removing any one of dilators 142A-D of surgical retractor insertion tool assembly 140 from incision 50 after surgical retractor 110 has been inserted in widened incision 50. Alternatively, any one of dilators 142A-D of surgical retractor insertion tool assembly 140 may remain in place for a portion or for the duration of the surgery.

While certain embodiments and details have been included herein for purposes of illustrating aspects of the instant disclosure, it will be apparent to those skilled in the art that various changes in systems, apparatus, and methods disclosed herein may be made without departing from the scope of the instant disclosure, which is defined, in part, in the appended claims. The words “including” and “having,” as used herein including the claims, shall have the same meaning as the word “comprising.”

What is claimed is:

1. A surgical retractor, comprising:
a retractor body having a first end with a first end opening, a second end opposite the first end and having a second end opening, and a pass through aperture extending through the retractor body from the first end opening to the second end opening, the first end opening having an elongate polygonal shape, and the second end opening having an elongate curved shape.

2. The surgical retractor as claimed in claim 1 wherein a central axis of the first end opening and a central axis of the second end opening are radially offset from each other.

3. The surgical retractor as claimed in claim 2 wherein the first end opening is sized larger than the second end opening.

4. The surgical retractor as claimed in claim 2 wherein the retractor body tapers from the first end to the second end.

5. The surgical retractor as claimed in claim 2 wherein the retractor body includes a chamfer at least one of the first end and the second end.

6. The surgical retractor as claimed in claim 2 further comprising a mounting bracket coupled to the retractor body proximate the first end of the retractor body.

7. The surgical retractor as claimed in claim 2 wherein the first end opening includes opposing lateral and medial sides and opposing cephalad and caudad sides, and at least a portion of a side surface of the second end opening is aligned with one of the lateral and cephalad sides when viewed from a top end of the retractor body.

8. The surgical retractor as claimed in claim 2 wherein the first end opening includes opposing lateral and medial sides and opposing cephalad and caudad sides, and the second end opening is laterally offset from at least one of the medial and caudad sides when viewed from a top end of the retractor body.

9. The surgical retractor as claimed in claim 2 wherein the first end opening has a major axis dimension and a minor axis dimension, and the second end opening has a major axis dimension and a minor axis dimension, wherein the major axis dimensions of the first and second end openings are arranged in parallel, and the minor axis dimensions of the first and second end openings are arranged in parallel.

10. The surgical retractor as claimed in claim 2 wherein the first end opening comprises a plurality of corners and at least one of the corners includes a tool retention loop.

11. The surgical retractor as claimed in claim 2 further comprising:
a surgical retractor insertion tool including a paddle-shaped spreader with a first end and a second end opposite the first end, and a handle removably coupled to the paddle-shaped spreader at the first end, the paddle-shaped spreader being sized to pass through the pass through aperture.

12. The surgical retractor as claimed in claim 2 further comprising:
a surgical retractor insertion tool including a plurality of dilators having different cross-sectional sizes, the plurality of dilators being sized to pass through the pass through aperture.

13. A surgical retractor, comprising:
a hollow retractor body having a first end opening defined at a first protrusion end, and a second end opening defined at a second insertion end opposite the first protrusion end, the first end opening having a polygonal shape and the second end opening having a curved shape;
wherein the second end opening is offset laterally from the first end opening.

14. The surgical retractor as claimed in claim 13 wherein the second end opening is offset longitudinally from the first end opening.

15. The surgical retractor as claimed in claim 13 wherein the polygonal shape is selected from the group consisting of a triangle, a square, a rectangle, a pentagon, a hexagon, a heptagon, an octagon, and a star having at least three points.

16. The surgical retractor as claimed in claim 13 wherein the curved shape is selected from the group consisting of a circle, an ellipse, a parabola, an oval, a hyperbolic curve, a pear-shaped curve, and a multi-lobed curve.

17. The surgical retractor as claimed in claim 13 wherein the first end opening is arranged in a first plane, and the second end opening is arranged in a second plane, the first and second planes being generally parallel.

18. A method of treating a patient, the method comprising:
making an incision in a patient;
inserting a surgical retractor into the incision, the surgical retractor comprising a hollow retractor body having a first end opening and a second end opening opposite the first end opening, the first end opening having a generally rectangular shape and the second end opening having a generally elliptical shape, the second end opening being offset laterally from the first end opening;
inserting an instrument through the surgical retractor and into the patient;
retaining the instrument in contact with a corner of the first end opening.

19. The method as claimed in claim 19 further comprising moving the instrument to a different corner of the first end opening to alter a relative angle between the second end opening and the instrument.

20. The method as claimed in claim 19 wherein inserting the surgical retractor includes inserting the surgical retractor in parallel with the incision.

21. The method as claimed in claim 19 further comprising:
inserting a surgical retractor insertion tool into the incision prior to inserting the surgical retractor into the incision, the surgical retractor insertion tool comprising at least one of a paddle-shaped spreader and a plurality of different sized dilators.

22. A retractor system, comprising:
a retractor body having a first end with a first end opening, a second end opposite the first end and having a second end opening, and a lumen extending through the retractor body from the first end opening to the second end opening, the first end opening having an elongate polygonal shape, and the second end opening having an elongate curved shape; and
an insertion tool having a handle and a coupling end, the coupling end having a second elongate shape adapted to engage the first end opening.

23. The retractor system as in claim 23, wherein the coupling end has an angled portion and a generally straight portion.

24. The retractor system as in claim 24 wherein the retractor has an angled side portion and a generally straight side portion.

25. The retractor system as in claim 25, wherein the angled portion of the coupling end is adapted to engage the angled side portion of the retractor.

26. The retractor system as in claim 23 wherein the second elongate shape defines an outer perimeter of the coupling end, the coupling end further having an inner edge in communication with the lumen.

27. The retractor system as in claim 27 wherein the coupling end inner edge has a shape that generally corresponds to the elongate curved shaped of the second end opening.

28. The retractor system as in claim 23 wherein the retractor body comprises a radiolucent material.
